(19) United States
(12) Patent Application Publication
(10) Pub. No.: US 2018/0322946 A1
(43) Pub. Date: Nov. 8, 2018

(54) Healthcare Actionable Intelligence Data Generation And Distribution

(71) Applicant: RxAdvance Corporation, Southborough, MA (US)

(72) Inventors: Ravi Venkata Ika, Southborough, MA (US); Madusudhana Rao Narahari, Westborough, MA (US); Prakash Surya Tallabattula, Northborough, MA (US); Anand Tati, Westborough, MA (US); Srinivas Gopaladasu, Shrewsbury, MA (US); Sobhan Babu Arigela, Westborough, MA (US); Kamal Bhagwanbhai Patel, Hopkinton, MA (US); Sreekanth Rao Belpu, Cary, NC (US)

(73) Assignee: RxAdvance Corporation

(21) Appl. No.: 15/969,796

(22) Filed: May 3, 2018

Related U.S. Application Data
(60) Provisional application No. 62/501,098, filed on May 4, 2017.

(51) Int. Cl. G16H 15/00; G16H 10/60; G06F 21/62

(52) U.S. Cl. CPC G16H 15/00; G06F 21/6254; G16H 10/60

(57) ABSTRACT

A method and a healthcare actionable intelligence data generation and distribution system (HAIDGDS) for securely generating and distributing healthcare actionable intelligence data to requesting entities in a computing environment are provided. The HAIDGDS identifies a patient from a healthcare eligibility request from one or more requesting entities. The HAIDGDS securely retrieves, compiles, stores, and transforms healthcare data sets of the patient from healthcare data sources into a unified data structure. The HAIDGDS determines overall patient health status and generates healthcare recommendations and alerts for the patient by analyzing the unified data structure including preexisting and ongoing healthcare data sets. The HAIDGDS generates a healthcare actionable intelligence report that includes the overall patient health status, the healthcare recommendations, and the alerts. The HAIDGDS generates and distributes a secure report access link with active session login information to access the healthcare actionable intelligence report to the requesting entities via a communication network.

### CROSS-REFERENCE TO RELATED APPLICATIONS

This application claims priority to and the benefit of the provisional patent application titled “Healthcare Actionable Intelligence Data Generation And Distribution”, application No. 62/501,098, filed in the United States Patent and Trademark Office on May 4, 2017. The specification of the above referenced patent application is incorporated herein by reference in its entirety.

### BACKGROUND

The method and the system disclosed herein, in general, relate to healthcare data processing in a healthcare environment. More particularly, the method and the system disclosed herein relate to securely generating and distributing healthcare actionable intelligence data to multiple healthcare entities in a computing environment, for example, a cloud computing environment or a peer to peer environment.

Conventional healthcare organizations, for example, pharmacy benefit management (PBM) organizations typically provide a limited set of information in a Health Insurance Portability and Accountability Act (HIPAA) X12 electronic data interchange (EDI) standard 271 benefit inquiry response in the form of, for example, a real time data stream response or a real time data stream file to any HIPAA X12 EDI standard 270 benefit inquiry request from a requesting entity as follows. A requesting entity, for example, a healthcare organization initiates a benefit inquiry request by submitting a HIPAA X12 EDI standard 270 benefit inquiry request directly to any processor or responding entity, for example, a PBM organization, or to a clearing house such as a third party administrator (TPA), an EDI vendor, etc., that maintains member or patient indices and request and response routing indices. The responding entity that receives this HIPAA X12 EDI standard 270 benefit inquiry request, populates minimum required data fields required to process the HIPAA X12 EDI standard 270 benefit inquiry request, and sends the HIPAA X12 EDI standard 271 benefit inquiry response back to the requesting entity or the clearing house. The clearing house, for example, the EDI vendor forwards the received HIPAA X12 EDI standard 271 benefit inquiry response from the responding entity to the requesting entity. The requesting entity uses a request handling system, for example, an electronic medical record (EMR) system or a practice management software (PMS) database to determine an eligible member's benefits. A healthcare entity, for example, a practitioner responsible for prescribing medication uses his/her practicing software to send the member's prescription to a point of sale (POS) location, for example, a pharmacy. When the member visits the POS location to collect the prescribed medication, the pharmacy dispenses the prescribed medication to the member after validating the member's benefits with the concerned responding entity. The minimum data fields populated by conventional responding entities are insufficient at a point of care (POC) to allow a practitioner to make an informed decision to send the member's prescription to the POS location. For example, when a requesting entity initiates the process of checking benefit and/or eligibility for a patient, a responding entity only provides an eligibility status. There is no mechanism for providing necessary actionable information back to the requesting entity. There is a need for populating all available data fields with available data comprising healthcare actionable intelligence data for use at the POC location, the POS location, etc., or by a plan sponsor case management team, the member or patient, etc., in a HIPAA X12 EDI standard 271 benefit inquiry response to the HIPAA X12 EDI standard 270 benefit inquiry request, to allow a practitioner responsible for prescribing the medication to make an informed decision based on the best available medical data to send the member's prescription to the POS location.

Hence, there is a long felt but unresolved need for a method and a system for securely aggregating healthcare data from multiple healthcare data sources, generating healthcare actionable intelligence data therefrom, and securely distributing the healthcare actionable intelligence data with overall patient health status and patient specific healthcare recommendations and alerts to multiple healthcare entities, for example, individual healthcare providers such as physicians, specialists, etc., healthcare provider organizations such as hospitals, clinics, etc., payers, prescribers, pharmacies, claim processing switches, pharmacy claim processors, coordination of benefits facilitators, financial transaction facilitators, third party administrators, Centers for Medicare and Medicaid Services (CMS), electronic data interchange vendors, plan sponsors, a plan sponsor case management team, any other healthcare data facilitators, etc., using advanced computing capability available in a computing environment, for example, a cloud computing environment or a peer to peer environment.

### SUMMARY OF THE INVENTION

This summary is provided to introduce a selection of concepts in a simplified form that are further disclosed in the detailed description of the invention. This summary is not intended to determine the scope of the claimed subject matter.

The method and the system disclosed herein address the above recited needs for securely aggregating healthcare data from multiple healthcare data sources, generating healthcare actionable intelligence data therefrom, and securely distributing the healthcare actionable intelligence data with overall patient health status and patient specific healthcare recommendations and alerts to multiple healthcare entities, for example, individual healthcare providers such as physicians, specialists, etc., healthcare provider organizations such as hospitals, clinics, etc., payers, prescribers, pharmacies, claim processing switches, pharmacy claim processors, coordination of benefits facilitators, financial transaction facilitators, third party administrators, Centers for Medicare and Medicaid Services (CMS), electronic data interchange vendors, plan sponsors, a plan sponsor case management team, any other healthcare data facilitators, etc., using advanced computing capability available in a computing environment, for example, a cloud computing environment or a peer to peer environment.

The method disclosed herein employs a healthcare actionable intelligence data generation and distribution system (HAIDGDS) comprising at least one processor configured to execute computer program instructions for securely generating and distributing healthcare actionable intelligence data to multiple requesting entities in a computing environment. The HAIDGDS receives a healthcare eligibility request from one or more requesting entities. The HAIDGDS identifies a patient from the received healthcare eligibility request. The HAIDGDS retrieves and compiles healthcare data sets of the identified patient from multiple healthcare data sources comprising, for example, precompiled existing data sources, via a secure electronic connectivity mode, for example, a secure electronic data interchange (EDI) connectivity mode. The HAIDGDS stores and transforms the retrieved and compiled healthcare data sets into a unified data structure. The HAIDGDS executes a specialized algorithm for determining overall patient health status and for generating healthcare recommendations and alerts for the identified patient by analyzing healthcare data contained in the unified data structure which is a repository of preexisting and ongoing healthcare data sets. The HAIDGDS generates a healthcare actionable intelligence report comprising the determined overall patient health status, the generated healthcare recommendations, and the generated alerts as a part of the healthcare actionable intelligence data. The HAIDGDS generates and distributes a secure report access link with active session login information to access the generated healthcare actionable intelligence report to one or more requesting entities via a communication network, for example, the world wide web, a peer to peer environment, the internet or private connections comprising cloud based communication mechanisms, etc., using one or more of multiple data exchange protocols.

In one or more embodiments, related systems comprise circuitry and/or programming for effecting the methods disclosed herein. The circuitry and/or programming can be any combination of hardware, software, and/or firmware configured to effect the methods disclosed herein depending upon the design choices of a system designer. Also, in an embodiment, various structural elements can be employed depending on the design choices of the system designer.

### BRIEF DESCRIPTION OF THE DRAWINGS

The foregoing summary, as well as the following detailed description of the invention, is better understood when read in conjunction with the appended drawings. For illustrating the invention, exemplary constructions of the invention are shown in the drawings. However, the invention is not limited to the specific methods and components disclosed herein. The description of a method step or a component referenced by a numeral in a drawing is applicable to the description of that method step or component shown by that same numeral in any subsequent drawing herein.

FIG. 1 illustrates a method for securely generating and distributing healthcare actionable intelligence data to requesting entities in a computing environment.

FIG. 2 exemplarily illustrates a flow diagram showing request and response processing performed by a healthcare actionable intelligence data generation and distribution system.

FIG. 3 exemplarily illustrates a flow diagram showing generation of healthcare actionable intelligence data related to patient health and pharmacy drug utilization from healthcare data sets by the healthcare actionable intelligence data generation and distribution system.

FIG. 4 exemplarily illustrates a system for securely generating and distributing healthcare actionable intelligence data related to patient health and pharmacy drug utilization to a point of care system.

FIG. 5 exemplarily illustrates a system for securely generating and distributing healthcare actionable intelligence data related to patient health and pharmacy drug utilization to a point of sale system.

FIG. 6 exemplarily illustrates a system for securely generating and distributing healthcare actionable intelligence data related to patient health and pharmacy drug utilization to a patient.

FIG. 7 exemplarily illustrates a system for securely generating healthcare actionable intelligence data using a unified data structure related to patient health and pharmacy drug advisory services.

FIG. 8 exemplarily illustrates a flow diagram showing generation of healthcare actionable intelligence data related to avoidable drug impacted medical costs from healthcare data sets by the healthcare actionable intelligence data generation and distribution system.

FIG. 9 exemplarily illustrates a system for securely generating and distributing healthcare actionable intelligence data related to avoidable drug impacted medical costs to external third party administrator systems.

FIG. 10 exemplarily illustrates a flow diagram showing an implementation of the healthcare actionable intelligence data generation and distribution system for securely generating and distributing healthcare actionable intelligence data related to patient health and pharmacy drug utilization to a wellness monitoring and reminder device and other wireless devices of a patient, for a follow up related to patient health and pharmacy drug utilization.

FIG. 11 exemplarily illustrates a system for securely generating and distributing healthcare actionable intelligence data to a point of sale system using a National Council for Prescription Drug Programs D.0 standard.

FIG. 12 exemplarily illustrates a system for securely generating and distributing healthcare actionable intelligence data to a point of care system for determining gaps in care for a patient.

FIG. 13 exemplarily illustrates a system for securely generating healthcare actionable intelligence data using a unified data structure for determining gaps in care for a patient.

FIG. 14 exemplarily illustrates a system for securely generating and distributing healthcare actionable intelligence data to a point of care system for predicting patient health status and recommending appropriate medical attention and/or care to the patient.

FIG. 15 exemplarily illustrates a system for securely generating healthcare actionable intelligence data using a unified data structure for predicting patient health status and recommending appropriate medical attention and/or care to the patient.

FIG. 16 exemplarily illustrates a system for securely generating healthcare actionable intelligence data using a unified data structure for predicting patient health status and recommending appropriate drugs to improve and/or maintain the patient health status.

FIG. 17 exemplarily illustrates a unified data structure for storing and aggregating healthcare data sets of a patient.

FIG. 18 exemplarily illustrates a screenshot of a healthcare actionable intelligence report generated by the healthcare actionable intelligence data generation and distribution system.

FIG. 19 exemplarily illustrates the system comprising the healthcare actionable intelligence data generation and distribution system for securely generating and distributing healthcare actionable intelligence data to requesting entities in a computing environment.

### DETAILED DESCRIPTION OF THE INVENTION

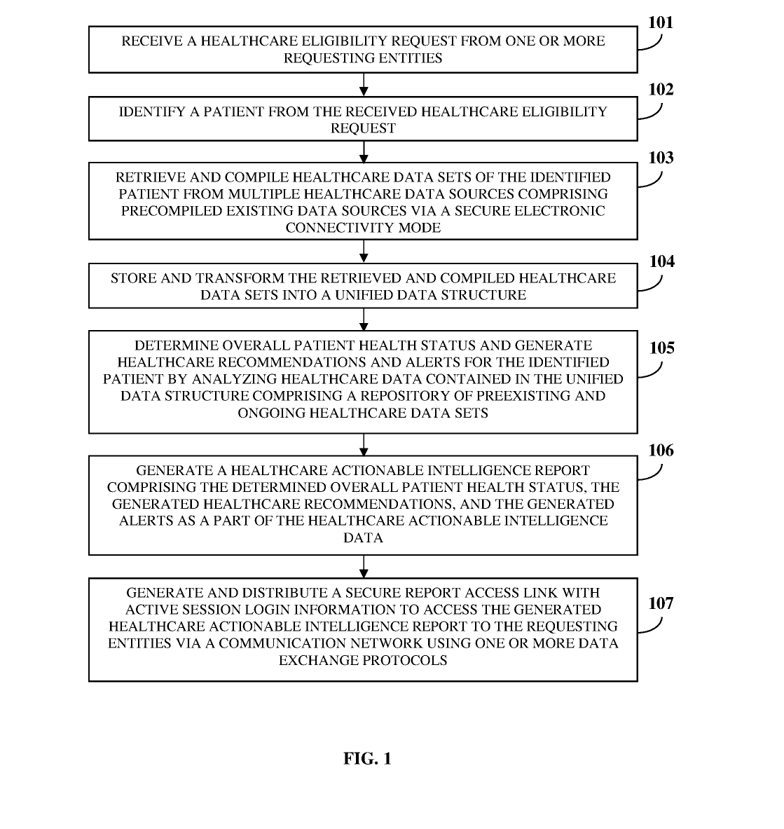

FIG. 1 illustrates a method for securely generating and distributing healthcare actionable intelligence data to requesting entities in a computing environment, for example, a cloud computing environment or a peer to peer environment. As used herein, “healthcare actionable intelligence data” refers to a data set containing insightful healthcare information that can be acted upon by different healthcare entities to draw conclusions, for example, about health of a patient, risks, etc., and that indicates further actions, for example, medical procedures and medications required to improve and/or maintain health status of the patient. Also, as used herein, “requesting entities” refer to individuals or entities that utilize the healthcare actionable intelligence data, for example, for making informed medical care decisions, for performing an informed healthcare and pharmacy drug care review to best manage the patient's health, for generating an informed healthcare and pharmacy drug care plan to best manage the patient's health while avoiding drug induced medical costs, for determining gaps in medical care, for predicting the patient's health status and recommending appropriate medical care and/or drugs to maintain and/or improve the patient's health status, etc. The requesting entities comprise, for example, seekers of pharmacy benefit, individual healthcare providers, healthcare provider organizations, payers, prescribers, specialists, pharmacies, claim processing switches, pharmacy claim processors, coordination of benefits facilitators, financial transaction facilitators, third party administrators, Centers for Medicare and Medicaid Services (CMS), electronic data interchange (EDI) vendors, plan sponsors such as employers, plan sponsor case and care management teams such as employer care management team staff, any other healthcare data facilitators, etc. Also, as used herein, “cloud computing environment” refers to a processing environment comprising configurable computing physical and logical resources, for example, software programs, networks, servers, storage media, virtual machines, applications, services, etc., and data distributed over a network, for example, the internet. The cloud computing environment provides on-demand network access to a shared pool of the configurable computing physical and logical resources. Also, as used herein, “peer to peer environment” refers to a network of computer systems that are connected to each other via a network, for example, the internet, where multiple files can be shared directly between computer systems on the network without the need for a central server. Each computer in a peer to peer environment operates as a file server and a client.

The method disclosed herein employs a healthcare actionable intelligence data generation and distribution system (HAIDGDS) comprising at least one processor configured to execute computer program instructions for securely generating and distributing healthcare actionable intelligence data to requesting entities, for example, healthcare entities in a computing environment. In an embodiment, the HAIDGDS is implemented as a web based platform hosted on a server or a network of servers accessible via a network, for example, the Internet, a wireless network, a mobile telecommunication network, etc. In another embodiment, the HAIDGDS comprises a software application downloadable and usable on a user device, for example, a personal computer, a mobile device, a smart phone, a tablet, a laptop, a personal digital assistant, a client device, a server, a portable electronic device, a network enabled computing device, an interactive network enabled communication device, etc., and configured to perform functions of the HAIDGDS. In another embodiment, one or more aspects of the HAIDGDS are performed on a client-server system that comprises components distributed among one or more server systems that perform multiple functions according to various embodiments. The HAIDGDS is not merely an electronic medical record (EMR) database consolidator. The HAIDGDS particularly facilitates a comprehensive drug utilization review (DUR) based on preexisting and ongoing pharmacy utilization details. The DUR is a structured, ongoing program that interprets patterns of drug utilization in relation to predetermined medically defined health and wellness criteria and attempts to prevent or minimize inappropriate prescribing of pharmaceutical drugs and medications to maintain the overall health of the patient.

In the method disclosed herein, the healthcare actionable intelligence data generation and distribution system (HAIDGDS) receives 101 a healthcare eligibility request from one or more requesting entities. For example, the HAIDGDS receives a Health Insurance Portability and Accountability Act (HIPAA) X12 electronic data interchange (EDI) standard 270 benefit inquiry request from a requesting entity directly or via a clearing house such as a third party administrator (TPA), an EDI vendor, etc. If the HIPAA X12 EDI standard 270 benefit inquiry request is performed through a clearing house, for example, an EDI vendor, the clearing house looks up a member registry table comprising details of multiple patients, also referred to as “members”, to forward the HIPAA X12 EDI standard 270 benefit inquiry request to the HAIDGDS. The HAIDGDS facilitates processing of an HIPAA EDI based eligibility request, for example, of a HIPAA Eligibility Transaction System (HETS) 270 standard, an eligibility response, for example, of a HETS 271 standard, and the National Council for Prescription Drug Programs (NCPDP) D.0 standard set for servicing healthcare entities, for example, the pharmacy industry and constituent entities of the pharmacy industry at a point of care (POC) and a point of sale (POS), for example, at hospitals or doctors' offices using an electronic medical record (EMR) system, a physician practice management system (PMS), health plans, one or more pharmacies, pharmacy benefit management (PBM) organizations, clearing houses, third party administrators (TPAs), EDI vendors, etc. As used herein, “point of sale (POS)” refers to a place or a location, for example, a pharmacy at which a retail transaction such as a pharmaceutical retail transaction is carried out. Also, as used herein, “point of care (POC)” refers to a place or a location where healthcare is provided to patients at a time of care. The POC locations comprise, for example, offices of individual providers such as medical doctors, primary care physician (PCP) offices, specialist offices, dentist offices, optometrist offices, healthcare provider organizations such as laboratories, skilled nursing facilities (SNFs), long term care (LTC) facilities, patient centered medical homes (PCMHs), accountable care organizations (ACOs), ambulatory care units, mental health centers, urgent care centers, addiction recovery centers, chiropractic clinics, integrated delivery systems (IDS), and other healthcare organizations, that have a need for patient eligibility, eligible medical benefits, eligible pharmacy benefits, and allied health information. “D.0” refers to an NCPDP and industry standard for pharmacy claims transactions.

The healthcare actionable intelligence data generation and distribution system (HAIDGDS) identifies 102 a patient from the received healthcare eligibility request. The HAIDGDS reads demographic information of the patient from data fields, for example, pharmacy data fields of the received healthcare eligibility request and identifies the patient. In an embodiment, the HAIDGDS does not require registration of the patient with the HAIDGDS. The HAIDGDS retrieves and compiles 103 healthcare data sets of the identified patient from multiple healthcare data sources comprising precompiled existing data sources via a secure electronic connectivity mode. As used herein, “healthcare data sets” refer to a collection of healthcare data of the patient stored, for example, in a single database table, or a single statistical data matrix, or in a collection of closely related tables. The healthcare data sets comprise, for example, medical records data sets, drug utilization data sets, physical vitals data sets, patient encounter data sets, patient pharmacy medication history, patient pharmacy medication plan adherence history, mental health records, etc. The medical records data sets comprise, for example, electronic medical records (EMRs), electronic health records (EHRs), personal medical records (PMRs), personal health records (PHRs), etc. The physical vitals sets comprise, for example, age, gender, height, weight, blood pressure values, heart rate, pulse rate, body temperature, blood oxygen levels, etc., and other health data required by a requesting entity to assess a patient's health status.

The healthcare data sets are of multiple formats comprising, for example, a Health Insurance Portability and Accountability Act (HIPAA) standard format such as an HIPAA 837 medical claim file in an X12 standards format, an HIPAA 834 membership file in an X12 standards format, an HIPAA 835 claim payment remittance advice file in an X12 standards format, etc., a National Council for Prescription Drug Programs (NCPDP) standard format such as the NCPDP D.0 standard, etc., open source, industry standard and custom electronic data interchange (EDI) formats such as customer formats, HAIDGDS medical formats, HAIDGDS pharmacy formats, etc. The HAIDGDS also retrieves and compiles healthcare data sets of, for example, a healthcare entity specified format, a user defined custom EDI format, etc. Also, as used herein, “healthcare data sources” refers to sources of data, for example, databases, database management systems, data warehouses, servers, etc., that store healthcare data, for example, in the form of healthcare data sets. The healthcare data sources comprise, for example, data sources of the requesting entities, health data sources, vision data sources, pharmacy data sources, dental data sources, patient vitals data sources, electronic medical records, electronic health records, personal medical records, personal health records, practice management systems, electronic prescription software, pharmacy benefit management systems, laboratory data sources, mental health data, medical claims data warehouse systems, patient encounter data sources, etc. The HAIDGDS searches the healthcare data sources for all available medical, pharmacy, dental and lab utilization data for the identified patient and retrieves and compiles the healthcare data sets for processing.

Consider an example where a patient enrolls into a health insurance plan. In this example, the healthcare actionable intelligence data generation and distribution system (HAIDGDS) receives demographic information of the patient and patient eligibility details as data files that are loaded into a database of the HAIDGDS. As the patient utilizes clinical services such as medical services, vision services, dental services, laboratory services, pharmaceutical services, etc., the HAIDGDS generates and processes insurance claims data. The HAIDGDS extracts and adds healthcare data sets with various types of information comprising, for example, diagnosis information, procedures, medications, laboratory information, and visit information to the patient's profile and thereafter stores the patient's profile identified by a unique identifier into a unified data structure as disclosed in the method step 104 below. The HAIDGDS analyzes the healthcare data sets algorithmically to identify actionable information and generates alerts such as therapeutic duplication, drug interactions, adverse drug events, high risk alerts, gaps in care, continuity of care, medication optimization, etc., as disclosed in the method step 105 below.

Also, as used herein, “secure electronic connectivity mode” refers to a mode of electronic communication for exchanging healthcare data via any secure electronic means. The secure electronic connectivity mode used by the healthcare actionable intelligence data generation and distribution system (HAIDGDS) for retrieving the healthcare data sets of the identified patient from the healthcare data sources is, for example, a secure electronic data interchange (EDI) connectivity mode. The EDI connectivity mode is a standard for a device to device interchange of formatted healthcare data via an electronic means. The EDI encompasses the entire EDI process comprising transmission, message flow, document format, and software used to interpret the healthcare data sets. The HAIDGDS is an automated data retrieval system that does not require personnel to manually enter healthcare data of the patient. The HAIDGDS implements automated processes that utilize proprietary and industry standard formats for gathering and uploading the healthcare data. The HAIDGDS imports the retrieved and compiled healthcare data sets via the secure EDI connectivity mode. The HAIDGDS develops multiple custom templates for different types of compiled healthcare data sets. The HAIDGDS parses the retrieved and compiled healthcare data sets by utilizing the developed custom templates. The HAIDGDS converts the parsed healthcare data sets into a machine readable format. The HAIDGDS reads and stores the converted healthcare data sets into a unified data structure. For purposes of illustration, the detailed description refers to the secure electronic connectivity mode being a secure EDI connectivity mode; however, the scope of the method and the HAIDGDS disclosed herein is not limited to the secure electronic connectivity mode being the secure EDI connectivity mode but may be extended to include other functionally equivalent secure electronic connectivity modes. A generic computer using a generic program cannot identify a patient from a healthcare eligibility request and cannot retrieve and compile healthcare data sets of the identified patient from multiple healthcare data sources via a secure electronic connectivity mode in accordance with the method steps disclosed above.

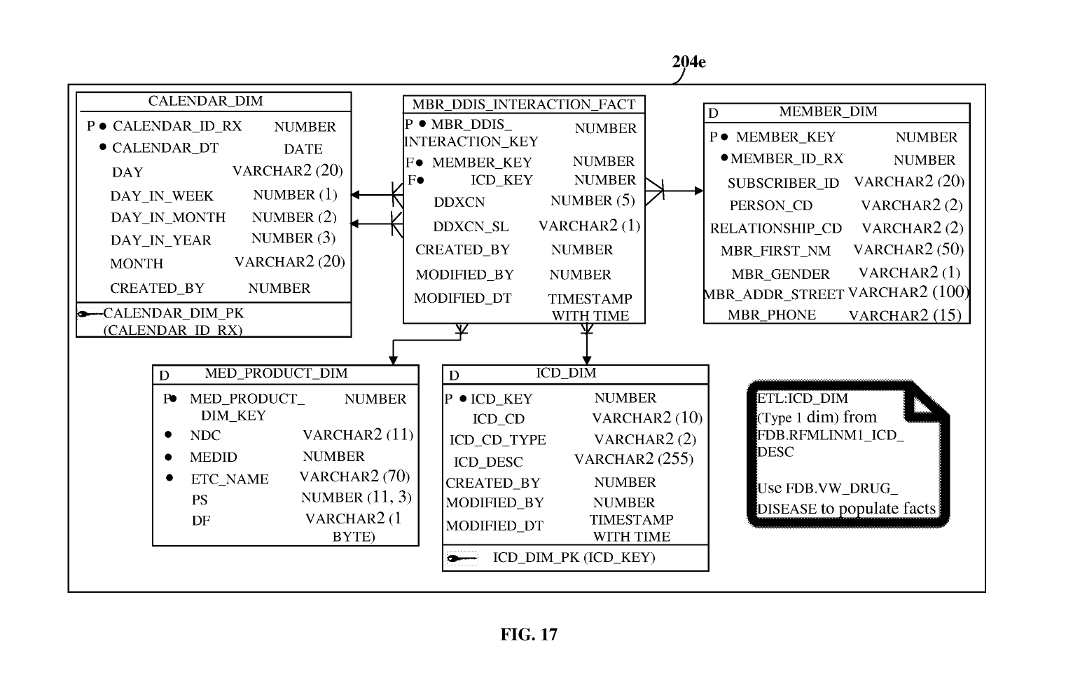

The healthcare actionable intelligence data generation and distribution system (HAIDGDS) stores and transforms 104 the retrieved and compiled healthcare data sets into a unified data structure as exemplarily illustrated in FIG. 17. The healthcare data contained in the unified data structure comprises, for example, demographic information of the identified patient with available contact information, health information of the identified patient, pharmacy medication history of the identified patient, pharmacy medication plan adherence history of the identified patient, mental health records, drug utilization data, physical vitals such as age, gender, height, weight, blood pressure values, pulse rate or heart beats per minute, body temperature, blood oxygen levels, etc., patient encounter data, medical diagnosis codes, medical procedure codes, financial codes, pharmacy national drug codes, financial transaction data associated with financial transactions such as copays, rebates, discounts, financial accumulators, subsidies, etc. The HAIDGDS structures the healthcare data sets into the unified data structure in a customized and normalized manner. One or more custom built translators are developed within the HAIDGDS to perform the transformation of the healthcare data sets from industry standard formats into the unified data structure. The HAIDGDS transforms the retrieved and compiled healthcare data sets into custom data models to configure and make the retrieved and compiled healthcare data sets compatible to underlying subsystems. For example, the HAIDGDS transforms pharmacy claims collected in an electronic data interchange (EDI) D.0 standard format into the unified data structure. In another example, the HAIDGDS transforms medical claims received in an American National Standards Institute (ANSI) Accredited Standards Committee (ASC) X12N 837 standard format into the unified data structure. The HAIDGDS uses the unified data structure to aggregate healthcare data comprising health information of the identified patient from the different healthcare data sources. A generic computer using a generic program cannot transform the retrieved and compiled healthcare data sets into a unified data structure in accordance with the method steps disclosed above.

The healthcare actionable intelligence data generation and distribution system (HAIDGDS) executes a specialized algorithm for determining 105 overall patient health status and generating healthcare recommendations and alerts for the identified patient by analyzing the healthcare data contained in the unified data structure comprising a repository of preexisting and ongoing healthcare data sets. As used herein, “patient health status” refers to state or condition of the patient's health. The overall patient health status comprises medication history such as a summary of all diagnosed, reported, and known medical conditions of the patient and medication alerts. The HAIDGDS determines the overall patient health status of the identified patient using medical diagnosis codes, medical procedure codes, financial codes, pharmacy national drug codes, financial transaction data associated with financial transactions such as copays, rebates, discounts, financial accumulators, subsidies, etc. For determining the overall patient health status, the HAIDGDS statistically analyzes population healthcare data to derive a relative health status of the patient. The specialized algorithm leverages the healthcare data comprising patient information, for example, number of emergency room visits, number of medications, length of stay in a hospital, severity of illness, identified gaps in care, and number of comorbid conditions, among other relevant information from the unified data structure to arrive at a normalized score that lies, for example, between 0 and 100, 0 being the lowest severity and 100 being the highest severity within a particular population group. Furthermore, where available, the HAIDGDS updates the normalized score more frequently by a home based vital measurements score and medication adherence scores. This normalized score provides a care giver with both the urgency of care required as well as appropriateness of a current regimen. The HAIDGDS compares the healthcare data with the repository of preexisting and ongoing healthcare data sets. The repository of preexisting and ongoing healthcare data sets comprises, for example, known high risk medical conditions, pharmacy drug interactions based on available utilization data and healthcare provider prescribed drugs yet to be fulfilled, best practices literature for managing ongoing patient health conditions, alternative lower cost pharmacy drug options, etc. A generic computer using a generic program cannot determine the overall patient health status of the identified patient and generate healthcare recommendations and alerts for the identified patient in accordance with the method steps disclosed above.

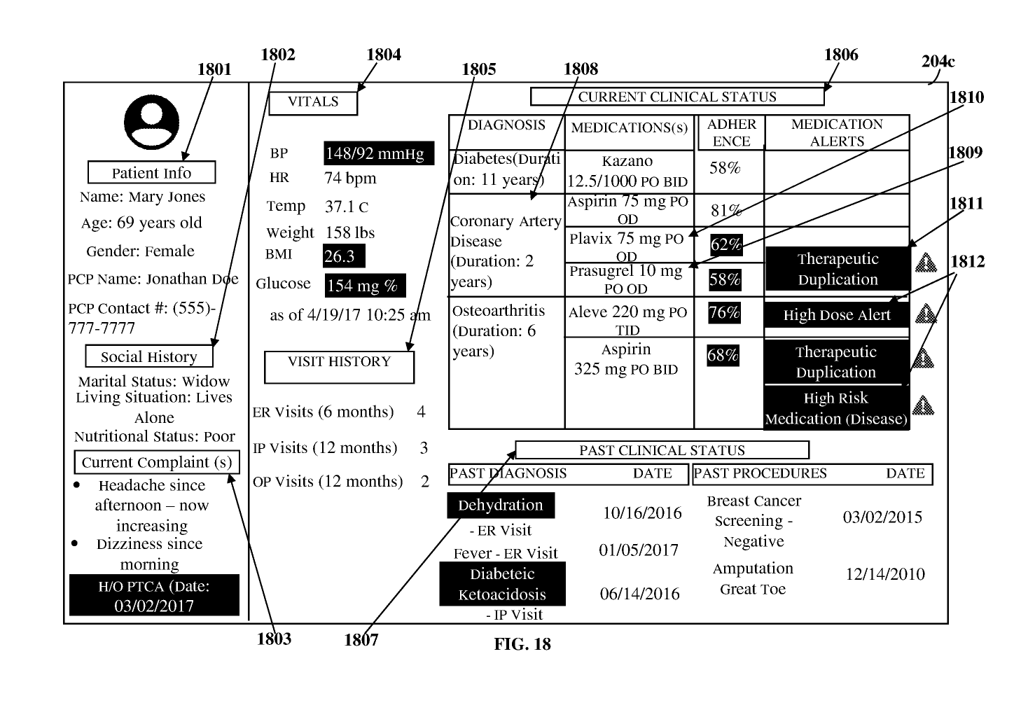

The healthcare actionable intelligence data generation and distribution system (HAIDGDS) generates 106 a healthcare actionable intelligence report comprising the determined overall patient health status, the generated healthcare recommendations, and the generated alerts as a part of the healthcare actionable intelligence data as exemplarily illustrated in FIG. 18. The HAIDGDS uses the unified data structure comprising the aggregated healthcare data sets imported from the healthcare data sources comprising, for example, data sources of federally recognized medical and health facilities, hospitals, clinics, doctors' offices, payer systems, doctors' practice management systems (PMSs), pharmacies, electronic prescription (e-prescription) software, pharmacy benefit management (PBM) systems, diagnostic laboratories, dentists' offices, optometrists' offices, physiotherapy centers, skilled nursing facilities, long term care facilities, patient centered medical homes, accountable care organizations, specialty care centers, electronic medical records, electronic health records, personal health records, medical claims data warehouse systems, financial facilitators, eligibility coordinators, etc., using Health Insurance Portability and Accountability Act (HIPAA) standard sets, the National Council for Prescription Drug Programs (NCPDP) standard sets, custom electronic data interchange (EDI) formats, etc., to generate the healthcare actionable intelligence report.

The healthcare actionable intelligence report is a human readable package of information for a requesting entity, for example, a healthcare provider to review at a point of care. The healthcare actionable intelligence report comprises healthcare actionable intelligence data of the patient, demographic information of the patient, contact information of the patient, a summary of all diagnosed, reported, and known medical conditions of the patient, a list of potential drug interactions, a list of lower cost alternative drugs available for prescription to the patient, a list of medical best practices to be considered for specific conditions that the patient is experiencing, a list of potential gaps in care of the patient identified by the healthcare actionable intelligence data generation and distribution system (HAIDGDS), etc. The healthcare actionable intelligence report further comprises, for example, day's supply information required to perform a comprehensive drug utilization review (DUR) analysis related to under usage and over usage of a particular drug, allowed fill numbers, fill or filled numbers required to perform a comprehensive adherence analysis for medication therapy management (MTM), compounding elements to determine a dispensing type of a claim, a service type for performing a detailed cost analysis related to retail versus long term care (LTC) versus specialty versus mail order, submission clarification codes for determining drug-disease related information, a true member incurred cost for performing the detailed cost analysis, etc. The healthcare actionable intelligence report further comprises information about the payer or healthcare insurer, examination findings associated with a health condition, symptoms of the health condition, diagnosis of the health condition, a prescribed treatment for the health condition, and healthcare data retrieved from the healthcare data sources. The HAIDGDS configured as a Health Insurance Portability and Accountability Act (HIPAA) compliant, dedicated actionable health intelligence reporting system draws upon all available healthcare data sources to formulate patient specific healthcare recommendations and alerts. The healthcare actionable intelligence report further comprises patient eligibility details, benefit plan details, formulary details, etc., among other healthcare actionable intelligence data for distribution to a point of care, a point of sale, plan sponsors, a plan sponsor case management team, any other healthcare data facilitators, and the identified patient. A generic computer using a generic program cannot generate the healthcare actionable intelligence report comprising the determined overall patient health status, the generated healthcare recommendations, and the generated alerts as a part of the healthcare actionable intelligence data in accordance with the method steps disclosed above.

The healthcare actionable intelligence data generation and distribution system (HAIDGDS) generates and distributes 107 a secure report access link with active session login information to access the generated healthcare actionable intelligence report to the requesting entities via a communication network, for example, the world wide web, the internet or private connections comprising cloud based communication mechanisms, a mobile cloud, an intranet, etc., using one or more data exchange protocols. The HAIDGDS distributes the secure report access link as a response, for example, a Health Insurance Portability and Accountability Act (HIPAA) X12 electronic data interchange (EDI) standard 271 benefit inquiry response to the healthcare eligibility request received from one or more of the requesting entities. The HAIDGDS populates all data fields of the HIPAA X12 EDI standard 271 benefit inquiry response with the available data and the healthcare actionable intelligence data in the healthcare actionable intelligence report. In an embodiment, the HAIDGDS selectively masks the healthcare actionable intelligence data in the healthcare actionable intelligence report to protect identity of the identified patient. The HAIDGDS encrypts, masks, or deletes some portions of the healthcare actionable intelligence data from view to protect and safeguard the identity of the identified patient based on the need for amount of information to be shared, privacy permissions, etc. The HAIDGDS finalizes and submits the healthcare actionable intelligence report with masked healthcare data to a messaging module of the HAIDGDS.

The messaging module of the healthcare actionable intelligence data generation and distribution system (HAIDGDS) creates an active, secure login for a time limited session with a unique one-time use token or a similar mechanism. The messaging module creates an active session using a single sign-on (SSO) or industry standard one-time use token web based security that is used to deliver the healthcare actionable intelligence report to the requesting entities. The messaging module creates a uniform resource identifier (URI) and embeds the active session login information for the healthcare actionable intelligence report into the URI. The messaging module creates an outbound URI submission wrapper. In an embodiment, the HAIDGDS delivers the healthcare actionable intelligence report to the requesting entities without a requirement for a dedicated application login or without a need for using a social networking platform. The secure report access link or the URI link to the healthcare actionable intelligence report is a secured clickable or copy-paste visitable URI that launches the HAIDGDS generated healthcare actionable intelligence report in one or more popup windows within a request handling system, for example, an electronic medical record (EMR) system, or a practice management system (PMS), or within a point of sale (POS) claim processing system, etc., of one or more requesting entities. The messaging module pushes the Health Insurance Portability and Accountability Act (HIPAA) compliant, secure URI link, for example, to a point of care (POC) system, a POS system, and/or the identified patient via a mobile cloud. A generic computer using a generic program cannot generate and distribute a secure report access link with active session login information to access the generated healthcare actionable intelligence report to the requesting entities in accordance with the method steps disclosed above. Moreover, a generic computer using a generic program cannot encrypt, mask, or delete portions of the healthcare actionable intelligence data from view to protect and safeguard the identity of the identified patient in accordance with the method steps disclosed above. Furthermore, a generic computer using a generic program cannot embed the active session login information for the healthcare actionable intelligence report into the URI in accordance with the method steps disclosed above.

The healthcare actionable intelligence data generation and distribution system (HAIDGDS) responds to the Health Insurance Portability and Accountability Act (HIPAA) X12 electronic data interchange (EDI) standard 270 benefit inquiry request with an HIPAA X12 EDI standard 271 benefit inquiry response in the healthcare actionable intelligence report to the requesting entity, for example, a healthcare organization, or the clearing house, for example, an EDI vendor. If the EDI vendor receives the HIPAA X12 EDI standard 271 benefit inquiry response, the EDI vendor forwards the HIPAA X12 EDI standard 271 benefit inquiry response to the healthcare organization. The HAIDGDS pushes and distributes the HIPAA X12 EDI standard 271 benefit inquiry response with the HIPAA compliant secure uniform resource identifier (URI) to a point of care (POC) system, a point of sale (POS) system, plan sponsors, a plan sponsor case management team, any other healthcare data facilitators, a payer or an employer care management team staff, and the patient via the mobile cloud. The proactive push of the healthcare actionable intelligence report to the requesting entities facilitates an early care coordination action to handle the patient's reported health conditions and expected health status changes based on a prognosis of a healthcare provider. The proactive early care coordination action assists in avoiding drug induced medical emergencies, resulting in cost savings for plan sponsors and the patient. The HAIDGDS distributes the URI and the response using one or more data exchange protocols. The data exchange protocols comprise, for example, a hypertext transfer protocol (HTTP), a secure hypertext transfer protocol (HTTPS), Winsock, a recommended standard number 232 (RS232) protocol, a file transfer protocol (FTP), a virtual private network protocol, and a secure file transfer protocol (SFTP). The healthcare actionable intelligence report is available to the requesting entities that have HIPAA agreements and authorized access to the healthcare data of the patient. In an embodiment, the HAIDGDS supports a single sign-on with any third party external identity management systems where required and/or requested.

The healthcare actionable intelligence data generation and distribution system (HAIDGDS) formulates and generates recommendations of drugs to administer to the patient by importing the patient's healthcare data from the healthcare data sources. Consider an example where a requesting entity, for example, a practitioner transmits a request to place an order for a prescription drug with a pharmacy based on the healthcare actionable intelligence report for drug delivery to the patient. Upon receiving the healthcare actionable intelligence report and upon review of the healthcare actionable intelligence data, the requesting entity at the point of care (POC) refers to a practicing software, for example, an electronic medical record (EMR) or available electronic prescribing (e-prescribing) software to make an informed decision based on the healthcare actionable intelligence data to send the patient's prescription to a point of sale (POS) location. When the patient visits the POS location to collect the prescribed medications, the POS location dispenses the prescribed medication to the patient after receiving a response for a submitted D.0 B1 transaction to the HAIDGDS. The HAIDGDS updates pharmacy claim record tables of the patient with information in a National Council for Prescription Drug Programs (NCPDP) D.0 pharmacy claim and transmits the updated information to a plan sponsor.

The data input, for example, the healthcare eligibility request from one or more requesting entities to the healthcare actionable intelligence data generation and distribution system (HAIDGDS) is technically transformed, processed, and executed by the specialized HAIDGDS algorithm as follows: The HAIDGDS receives the healthcare eligibility request, reads demographic information of the patient from the data fields, and identifies the patient. The HAIDGDS retrieves healthcare data sets of the identified patient from multiple healthcare data sources via the secure electronic connectivity mode. The HAIDGDS searches the healthcare data sources for all available medical, pharmacy, dental and laboratory utilization data of the identified patient and compiles the healthcare data sets for processing. The HAIDGDS receives and stores the retrieved and compiled healthcare data sets into database tables of the HAIDGDS. The HAIDGDS then technically transforms, processes, and executes the stored healthcare data sets into the unified data structure using the HAIDGDS algorithm. The HAIDGDS analyzes the healthcare data stored in the unified data structure and determines overall patient health status. The HAIDGDS also generates healthcare recommendations and alerts for the identified patient. The HAIDGDS generates the healthcare actionable intelligence report comprising the determined overall patient health status, the generated healthcare recommendations, and the generated alerts as a part of the healthcare actionable intelligence data. The HAIDGDS selectively masks the healthcare actionable intelligence data in the healthcare actionable intelligence report. The HAIDGDS generates the secure report access link with the active session login information to access the generated healthcare actionable intelligence report, for distribution to the requesting entities via the communication network. The HAIDGDS therefore utilizes the specialized HAIDGDS algorithm to transform the healthcare eligibility request into the secure report access link that provides active session login information to access the generated healthcare actionable intelligence report.

The method and the healthcare actionable intelligence data generation and distribution system (HAIDGDS) disclosed herein provide an improvement in healthcare data processing in healthcare environment computer related technology as follows. The HAIDGDS retrieves healthcare data sets of the identified patient from multiple healthcare data sources comprising precompiled existing data sources using a secure electronic connectivity mode. The HAIDGDS searches the healthcare data sources for all available medical, pharmacy, dental and lab utilization data for the identified patient and securely aggregates the healthcare data sets. The HAIDGDS stores and transforms the securely aggregated healthcare data sets into the unified data structure utilizing the HAIDGDS algorithm. The HAIDGDS determines the overall patient health status, generates healthcare recommendations and alerts for the identified patient, generates a healthcare actionable intelligence report, and securely distributes a secure report access link to access the healthcare actionable intelligence report with patient specific healthcare recommendations and alerts to multiple healthcare entities via the communication network. The HAIDGDS determines potential gaps in care of the identified patient using the HAIDGDS algorithm and avoids additional drug induced medical care needed by prescribing pharmacy drugs that are safe to use in combination with other existing prescription drugs. The HAIDGDS populates all the available data fields, for example, in a Health Insurance Portability and Accountability Act (HIPAA) X12 electronic data interchange (EDI) standard 271 benefit inquiry response to a HIPAA X12 EDI standard 270 benefit inquiry request, with the data available comprising healthcare actionable intelligence data for use at a point of care (POC) location, a point of sale (POS) location, a plan sponsor case management team, or for use by the patient, etc. The HAIDGDS generates a time sensitive secure report access link, for example, a user clickable uniform resource indicator (URI) active hyperlink in response to the healthcare eligibility request from the requesting entities. The secure report access link, when accessed, provides the healthcare actionable intelligence data comprising, for example, clinical information, utilization information, risk alerts, etc., about the patient, that is, more than just an eligibility status, for use, for example, at the point of care location in decision making during the patient's visit. The healthcare actionable intelligence report generated by the HAIDGDS particularly facilitates a comprehensive drug utilization review (DUR) based on preexisting and ongoing pharmacy utilization details.

In the method disclosed herein, the design and the flow of interactions between the healthcare actionable intelligence data generation and distribution system (HAIDGDS), multiple healthcare data sources, a point of care, a point of sale, a user device of the patient, and a third party administrator or system are deliberate, designed, and directed. Every healthcare eligibility request received from requesting entities is configured by the HAIDGDS to generate a set of predictable outcomes. The HAIDGDS implements one or more specific computer programs to direct the received healthcare eligibility request towards a set of end results. The interactions designed by the HAIDGDS allow the HAIDGDS to integrate and communicate with healthcare data sources via a secure electronic connectivity mode for retrieving and compiling healthcare data sets of the identified patient and transforming the healthcare data sets into the unified data structure, and from this content, through the use of other, separate and autonomous computer programs, determine the overall patient health status and generate healthcare recommendations and alerts. This determination is used to trigger generation of a healthcare actionable intelligence report and generation and distribution of a secure report access link with active session login information to access the generated healthcare actionable intelligence report to the requesting entities. To identify the patient from the received healthcare eligibility request, retrieve and compile healthcare data sets of the identified patient from multiple healthcare data sources, store and transform the retrieved and compiled healthcare data sets into the unified data structure, determine overall patient health status and generate healthcare recommendations and alerts for the identified patient by analyzing healthcare data contained in the unified data structure comprising the repository of preexisting and ongoing healthcare data sets, generate the healthcare actionable intelligence report, and generate and distribute the secure report access link with active session login information to access the generated healthcare actionable intelligence report requires six or more separate computer programs and subprograms, the execution of which cannot be performed by a person using a generic computer with a generic program.

The focus of the method and the healthcare actionable intelligence data generation and distribution system (HAIDGDS) disclosed herein is on an improvement to the computer functionality itself, and not on economic or other tasks for which a generic computer is used in its ordinary capacity. Accordingly, the method and the HAIDGDS disclosed herein are not directed to an abstract idea. Rather, the method and the HAIDGDS disclosed herein are directed to a specific improvement to the way the computing system of the HAIDGDS operates, embodied in, for example, identifying a patient from the received healthcare eligibility request, retrieving and compiling healthcare data sets from multiple healthcare data sources, storing and transforming the retrieved and compiled healthcare data sets into the unified data structure, determining overall patient health status of the identified patient, generating healthcare recommendations and alerts for the identified patient, generating a healthcare actionable intelligence report comprising healthcare actionable intelligence data, generating the secure report access link with active session login information to access the generated healthcare actionable intelligence report, and distributing the secure report access link to multiple requesting entities via the communication network using multiple data exchange protocols.

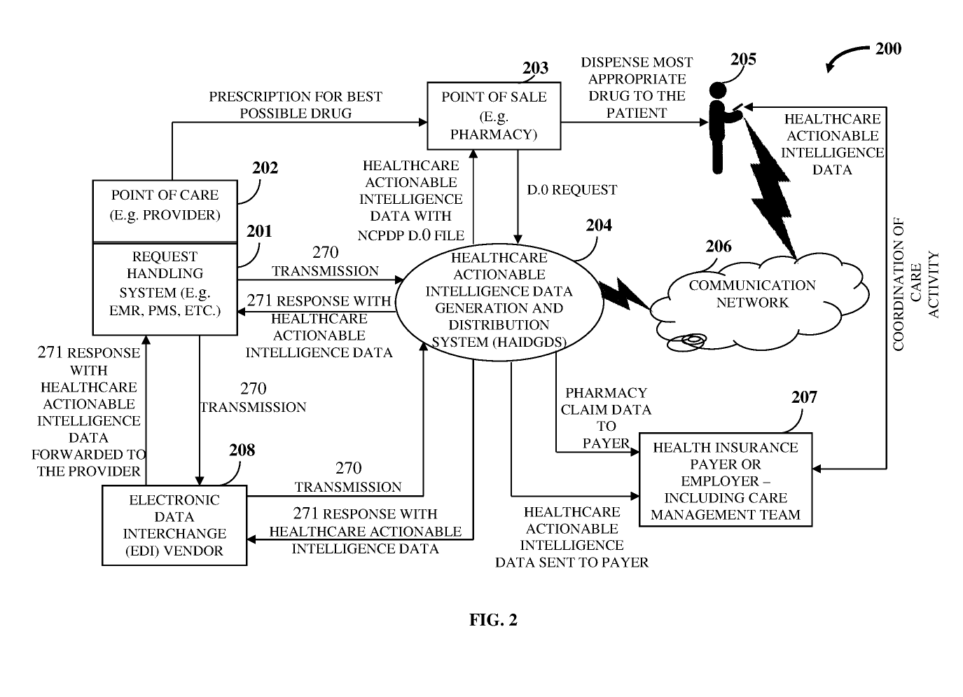

FIG. 2 exemplarily illustrates a flow diagram showing request and response processing performed by the healthcare actionable intelligence data generation and distribution system (HAIDGDS) 204. The HAIDGDS 204 executes a specialized computer system algorithm for performing request and response processing. In the system 200 disclosed herein, the HAIDGDS 204 is configured, for example, as a collaborative pharmacy benefit management platform. The HAIDGDS 204 receives a Health Insurance Portability and Accountability Act (HIPAA) X12 electronic data interchange (EDI) standard 270 benefit inquiry request from a request handling system 201, for example, an electronic medical record (EMR) system or a practice management system (PMS) of the requesting entity, for example, a provider, a physician, or a healthcare organization. In an embodiment, the HAIDGDS 204 receives the HIPAA X12 EDI standard 270 benefit inquiry request through a switch or an EDI vendor 208. The EDI vendor 208 looks up a member registry table to forward the HIPAA X12 EDI standard 270 benefit inquiry request to the HAIDGDS 204. Upon receiving the HIPAA X12 EDI standard 270 benefit inquiry request, the HAIDGDS 204 populates pharmacy data fields of the HIPAA X12 EDI standard 271 benefit inquiry response with the healthcare actionable intelligence data and transmits the HIPAA X12 EDI standard 271 benefit inquiry response to the requesting entity or to the EDI vendor 208 based on the origin of the HIPAA X12 EDI standard 270 benefit inquiry request. The HIPAA X12 EDI standard 271 benefit inquiry response comprises, for example, patient eligibility details, benefit plan details, formulary details, and the healthcare actionable intelligence data.

The healthcare actionable intelligence data generation and distribution system (HAIDGDS) 204 pushes the healthcare actionable intelligence data via a secure visitable uniform resource identifier (URI) that launches components of the HAIDGDS 204 to a point of care (POC) 202, for example, a provider, a point of sale (POS) 203, for example, a pharmacy, etc., a plan sponsor management team, a payer or employer care management team staff 207, and a member or patient 205 via a communication network 206. If the electronic data interchange (EDI) vendor 208 receives the Health Insurance Portability and Accountability Act (HIPAA) X12 EDI standard 271 benefit inquiry response along with the healthcare actionable intelligence data, the EDI vendor 208 forwards the HIPAA X12 EDI standard 271 benefit inquiry response with the healthcare actionable intelligence data to the requesting entity. A provider at the POC 202 reviews the healthcare actionable intelligence data, prescribes medication by referring to the request handling system 201 and an electronic prescribing software to make an informed decision about the available medical data, and sends an electronic or paper based prescription of the patient 205 to the POS 203. The POS 203 dispenses medication, that is, the most appropriate drug to the patient 205. The HAIDGDS 204 transmits healthcare actionable intelligence data with a National Council for Prescription Drug Programs (NCPDP) D.0 file to a D.0 request of the POS 203. The HAIDGDS 204 also transmits healthcare actionable intelligence data to a payer, for example, a health insurance payer, employer, or care management team 207. The HAIDGDS 204 updates the patient record tables with updated information submitted in a D.0 pharmacy claim and forwards the D.0 pharmacy claim to the payer, employer, or care management team 207, that coordinates care activity with the patient 205.

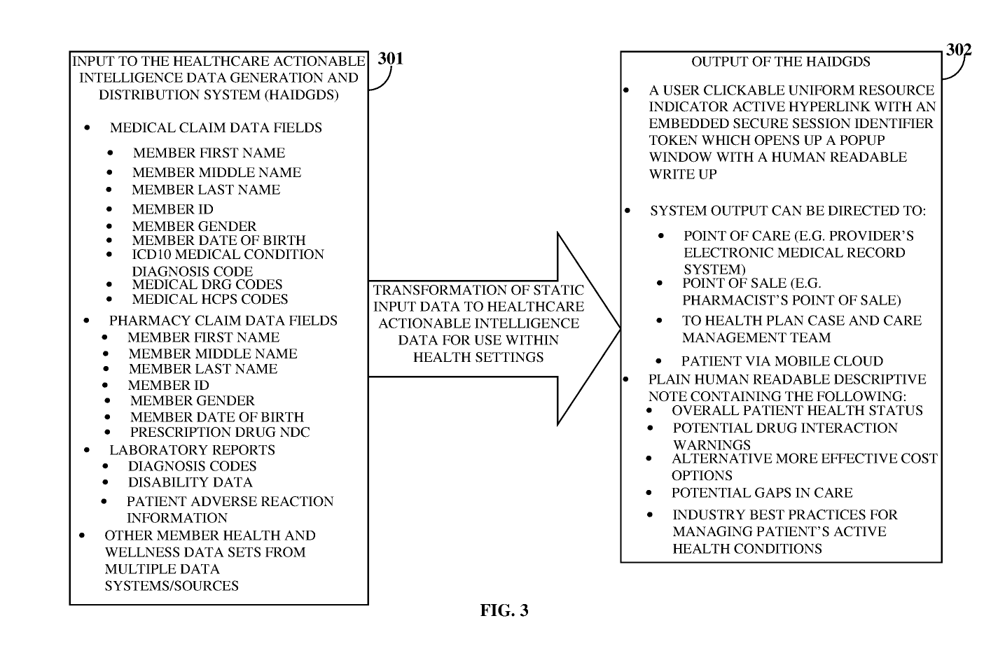

FIG. 3 exemplarily illustrates a flow diagram showing generation of healthcare actionable intelligence data related to patient health and pharmacy drug utilization from healthcare data sets by the healthcare actionable intelligence data generation and distribution system (HAIDGDS) 204 exemplarily illustrated in FIG. 2. The HAIDGDS 204 executes a specialized algorithm for generating the healthcare actionable intelligence data related to patient health and pharmacy drug utilization from the healthcare data sets. The HAIDGDS 204 receives input healthcare data 301 comprising, for example, medical claim data fields, pharmacy claim data fields, laboratory reports, and other member health and wellness data sets from multiple data systems or data sources. As used herein, the terms “member” and “patient” are used interchangeably and refer to an individual such as a person who requires healthcare. The medical claim data fields comprise, for example, a member first name data field, a member middle name data field, a member last name data field, a member identifier (ID) data field, a member gender data field, a member date of birth data field, an international classification of diseases, edition 10 (ICD10) or the latest ICD classification standards medical condition diagnosis codes data field, a medical diagnosis related group (DRG) codes data field, and a medical healthcare common procedure coding system (HCPS) codes data field. The pharmacy claim data fields comprise, for example, a member first name data field, a member middle name data field, a member last name data field, a member ID data field, a member gender data field, a member date of birth data field, and a prescription drug national drug code (NDC) data field. The laboratory reports comprise, for example, diagnosis codes, disability data, and patient adverse reaction information. The healthcare data sets comprising member health, financial, and wellness data sets from multiple data systems or multiple healthcare data sources are disclosed in the detailed description of FIG. 1.

The healthcare actionable intelligence data generation and distribution system (HAIDGDS) 204 executes the specialized algorithm for transforming the static input healthcare data 301 to the healthcare actionable intelligence data contained in the healthcare actionable intelligence report for use within health settings. The output 302 of the HAIDGDS 204 comprises a user clickable uniform resource indicator (URI) active hyperlink with an embedded secure session identifier token which opens up a popup window with a human readable package of information contained in the healthcare actionable intelligence report. The human readable package of information comprises, for example, an overall patient health status, potential drug interaction warnings, alternative more cost effective drug options, potential gaps in care, health statements, notes, and industry best practices for managing the patient's or member's active health condition. The HAIDGDS 204 distributes the healthcare actionable intelligence report to a point of care (POC) 202, that is, the request handling system 201, for example, an electronic medical record (EMR) system, a pharmacist's point of sale (POS) 203, a health plan case and care management team 207, and the patient 205 through the user clickable URI active hyperlink, for example, via a mobile cloud as exemplarily illustrated in FIG. 2.

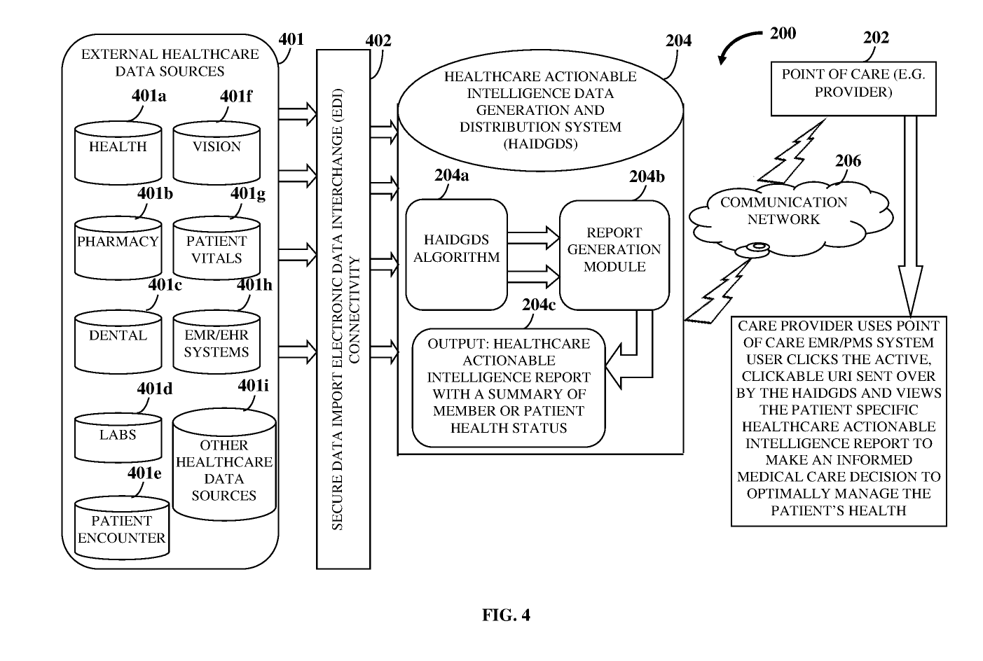

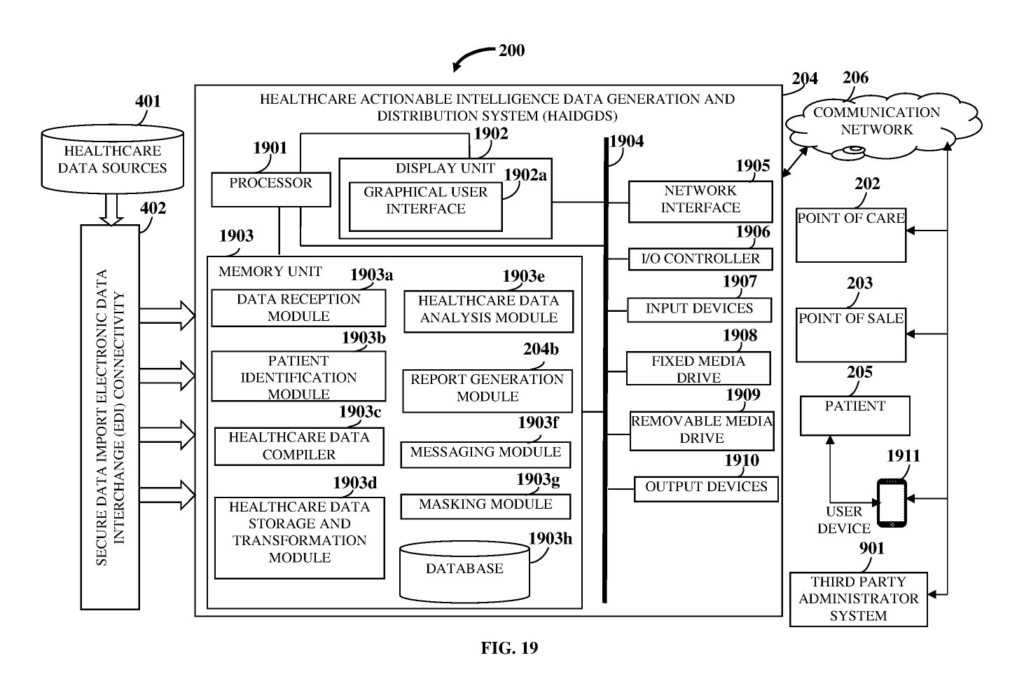

FIG. 4 exemplarily illustrates a system 200 for securely generating and distributing healthcare actionable intelligence data related to patient health and pharmacy drug utilization to a requesting entity, for example, a point of care (POC) system 202 of a healthcare provider. The healthcare actionable intelligence data generation and distribution system (HAIDGDS) 204 imports healthcare data sets of a patient from multiple healthcare data sources 401 comprising, for example, health data sources 401a, pharmacy data sources 401b, dental data sources 401c, laboratory data sources 401d, patient encounter data sources 401e, vision data sources 401f, patient vitals data sources 401g, electronic medical record (EMR) and/or electronic health record (EHR) systems 401h, and other healthcare data sources 401i via a secure electronic data interchange (EDI) connectivity mode, herein referred to as a secure data import EDI connectivity mode 402. The HAIDGDS 204 executes the HAIDGDS algorithm 204a and a report generation module 204b of the HAIDGDS 204 for securely generating a healthcare actionable intelligence report 204c comprising healthcare actionable intelligence data related to patient health and pharmacy drug utilization. The HAIDGDS 204 transmits the generated healthcare actionable intelligence report 204c to the POC system 202 via a secure report access link. In an embodiment, the report generation module 204b in communication with a healthcare data analysis module 1903e of the HAIDGDS 204 exemplarily illustrated in FIG. 19, determines a dispensing type of a claim from compounding elements. In another embodiment, the report generation module 204b in communication with the healthcare data analysis module 1903e determines drug disease related information using submission clarification codes. The report generation module 204b transforms the imported healthcare data sets in the unified data structure into the healthcare actionable intelligence report 204c, which is a comprehensive summary report for the requesting entity's review and decision support as disclosed in the detailed description of FIG. 1. The healthcare actionable intelligence report 204c comprises a summary of the patient's health status. The HAIDGDS 204 publishes the secure report access link, that is, an active, clickable uniform resource identifier (URI) as a Health Insurance Portability and Accountability Act (HIPAA) Eligibility Transaction System (HETS) 271 response to the requesting entity's EMR and/or practice management software (PMS) POC system 202 via the communication network 206, for example, the internet. The requesting entity, for example, a care provider clicks the active, clickable URI at the POC system 202 and views a popup window with the healthcare actionable intelligence report 204c as delivered by the HAIDGDS 204. The requesting entity reviews the healthcare actionable intelligence data comprising the overall patient health status, the recommendations, and the alerts among other healthcare actionable intelligence data contained in the patient specific healthcare actionable intelligence report 204c to make an informed medical care decision to optimally manage the patient's health, and determines the next steps to provide optimal medical care to the patient, which include avoiding potential drug induced medical events.

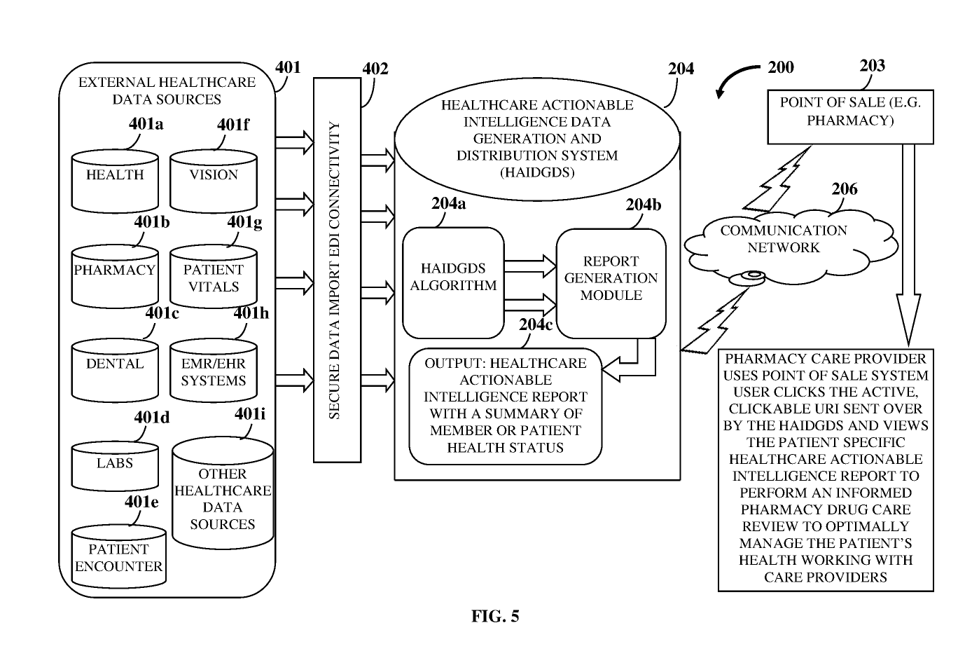

FIG. 5 exemplarily illustrates a system 200 for securely generating and distributing healthcare actionable intelligence data related to patient health and pharmacy drug utilization to a requesting entity, for example, a point of sale (POS) system 203 of a pharmacy or pharmacist. The healthcare actionable intelligence data generation and distribution system (HAIDGDS) 204 imports healthcare data sets of a patient from multiple healthcare data sources 401 via a secure data import electronic data interchange (EDI) connectivity mode 402 as disclosed in the detailed description of FIG. 4. The HAIDGDS 204 executes the HAIDGDS algorithm 204a and the report generation module 204b to transform the imported healthcare data sets in the unified data structure into the healthcare actionable intelligence report 204c, which is a comprehensive summary report of the patient's health status for the requesting entity's review and decision support as disclosed in the detailed description of FIG. 1. The HAIDGDS 204 publishes an active, clickable uniform resource identifier (URI) to the requesting entity's POS system 203 via the communication network 206, for example, the internet. The requesting entity, for example, a pharmacy care provider clicks the active, clickable URI at the POS system 203 and views a popup window with the healthcare actionable intelligence report 204c as delivered by the HAIDGDS 204. The requesting entity reviews the healthcare actionable intelligence data comprising the overall patient health status, the recommendations, and the alerts among other healthcare actionable intelligence data contained in the patient specific healthcare actionable intelligence report 204c to perform an informed pharmacy drug care review to optimally manage the patient's health, and determines the next steps to provide optimal medical care to the patient by collaborating with requesting entities, for example, care providers at the point of care (POC) system 202 exemplarily illustrated in FIG. 2 and FIG. 4.

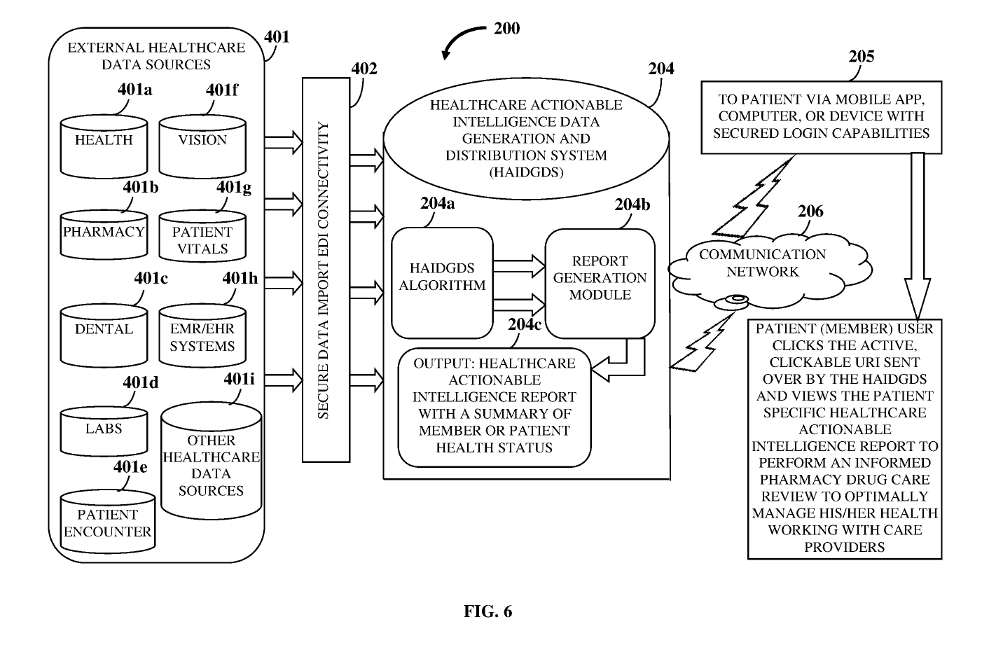

FIG. 6 exemplarily illustrates a system 200 for securely generating and distributing healthcare actionable intelligence data related to patient health and pharmacy drug utilization to a patient, for example, via a mobile application, a computer, or a device with secured login capabilities through a communication network 206, for example, a mobile cloud. The healthcare actionable intelligence data generation and distribution system (HAIDGDS) 204 imports healthcare data sets of a patient from multiple healthcare data sources 401 via a secure data import electronic data interchange (EDI) connectivity mode 402 as disclosed in the detailed description of FIG. 4. The HAIDGDS 204 executes the HAIDGDS algorithm 204a and the report generation module 204b transform the imported healthcare data sets in the unified data structure into the healthcare actionable intelligence report 204c, which is a comprehensive summary report of the patient's health status for a requesting entity's, for example, a healthcare provider's review and decision support as disclosed in the detailed description of FIG. 1. The HAIDGDS 204 publishes an active, clickable uniform resource identifier (URI) to the patient via the communication network 206, for example, via the mobile application, the computer, or the device with secured login capabilities. The patient clicks the active, clickable URI and views a popup window with the healthcare actionable intelligence report 204c as delivered by the HAIDGDS 204. The patient reviews the patient specific healthcare actionable intelligence report 204c and performs an informed pharmacy drug care review to optimally manage his/her health by collaborating with the healthcare providers at the point of care (POC) system 202 exemplarily illustrated in FIG. 2 and FIG. 4. The patient reviews and discusses the healthcare actionable intelligence data, further actions, and recommendations with the healthcare providers at the POC system 202. The patient discusses the next steps in receiving optimal medical care with his/her healthcare providers and may make selective lifestyle changes to better manage the health conditions identified in the healthcare actionable intelligence report 204c based on the discussions with the healthcare providers.

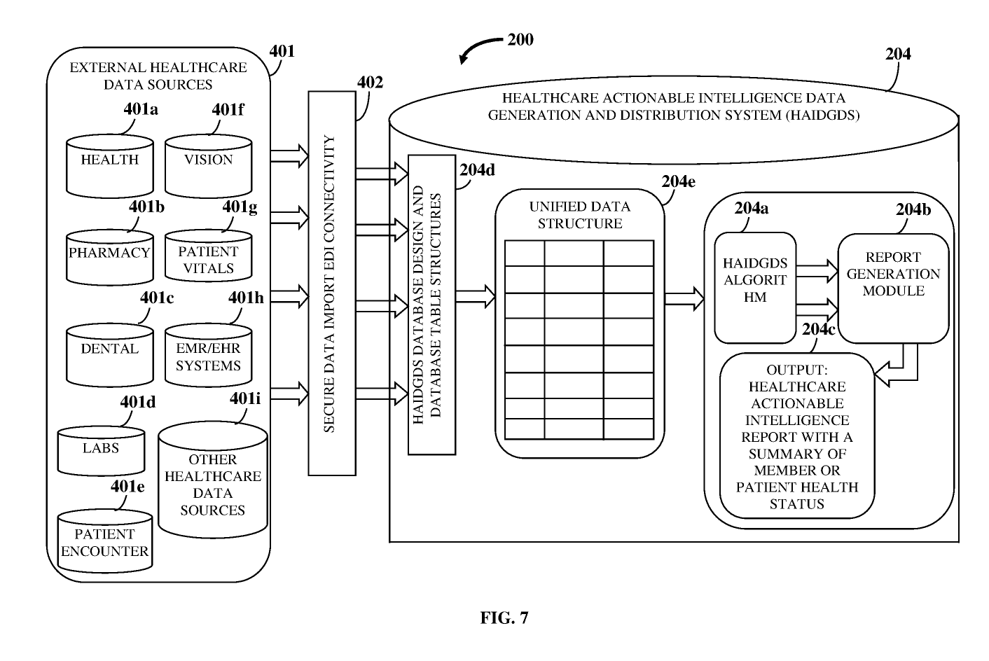

FIG. 7 exemplarily illustrates a system 200 for securely generating healthcare actionable intelligence data using a unified data structure 204e related to patient health and pharmacy drug advisory services. The healthcare actionable intelligence data generation and distribution system (HAIDGDS) 204 imports healthcare data sets of a patient from multiple healthcare data sources 401 via a secure data import electronic data interchange (EDI) connectivity mode 402 as disclosed in the detailed description of FIG. 4. The HAIDGDS 204 implements database design and database table structures 204d and a unified data structure 204e comprising unified database data tables. The HAIDGDS database design and database table structures 204d are used to configure the unified data structure 204e. The HAIDGDS 204 transforms the imported healthcare data sets into the unified data structure 204e as disclosed in the detailed description of FIG. 1. The HAIDGDS 204 analyzes the healthcare data contained in the unified data structure 204e by executing the HAIDGDS algorithm 204a and executes the report generation module 204b for generating the healthcare actionable intelligence report 204c for the requesting entity's review, for example, the healthcare provider's review and decision support as disclosed in the detailed description of FIG. 1. The healthcare actionable intelligence report 204c comprises a summary of the patient's health status.

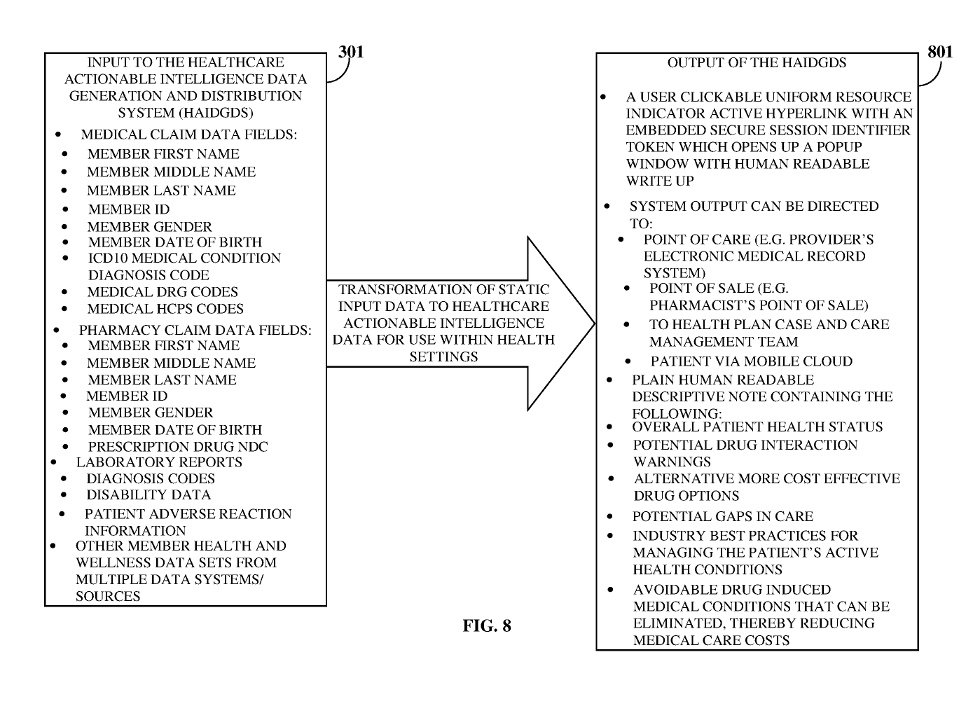

FIG. 8 exemplarily illustrates a flow diagram showing generation of healthcare actionable intelligence data related to avoidable drug impacted medical costs from healthcare data sets by the healthcare actionable intelligence data generation and distribution system (HAIDGDS) 204 exemplarily illustrated in FIG. 2. The HAIDGDS 204 receives input healthcare data 301 as disclosed in the detailed description of FIG. 3. The HAIDGDS 204 executes the HAIDGDS algorithm 204a for transforming the static input healthcare data 301 into the healthcare actionable intelligence data contained in the healthcare actionable intelligence report 204c exemplarily illustrated in FIGS. 4-7, FIG. 9, FIGS. 11-16, and FIG. 18, for use within health settings. The output 801 of the HAIDGDS 204 comprises a user clickable uniform resource indicator (URI) active hyperlink with an embedded secure session identifier token which opens up a popup window with a human readable package of information contained in the healthcare actionable intelligence report 204c. The human readable package of information comprises, for example, an overall patient health status, potential drug interaction warnings, alternative more cost effective drug options, potential gaps in care, industry best practices for managing the patient's active health conditions, potential gaps in member care, and avoidable drug induced medical conditions that can be eliminated, thereby reducing medical care costs. The HAIDGDS 204 distributes the healthcare actionable intelligence report 204c to one or more of a point of care (POC) system 202, for example, the provider's electronic medical record (EMR) system, a point of sale (POS) system 203, for example, the pharmacist's POS system, a health plan case and care management team 207, and the patient 205 via a mobile cloud as exemplarily illustrated in FIG. 2.

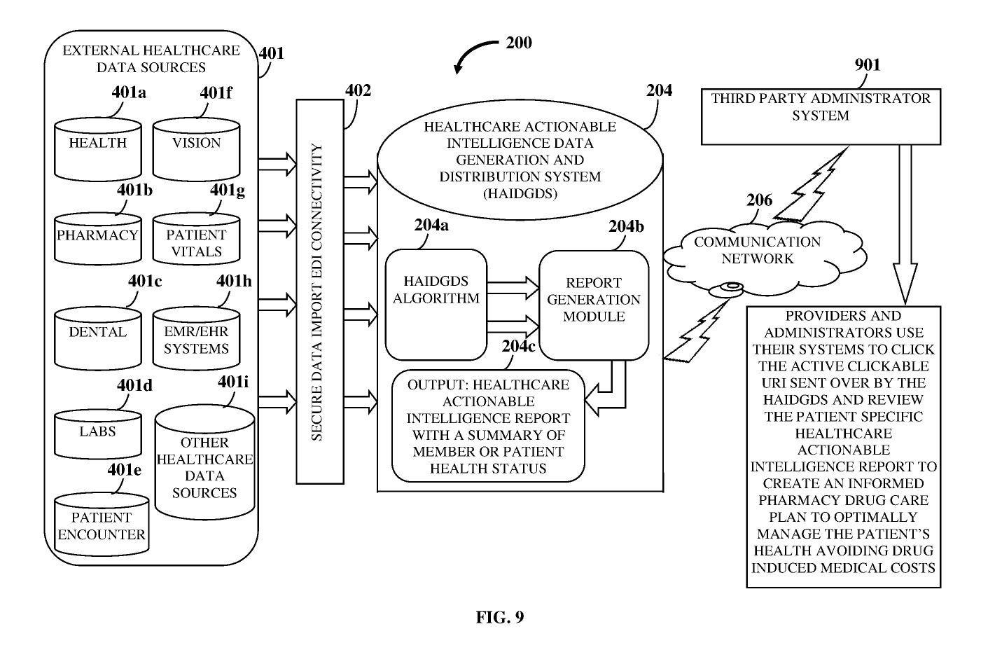

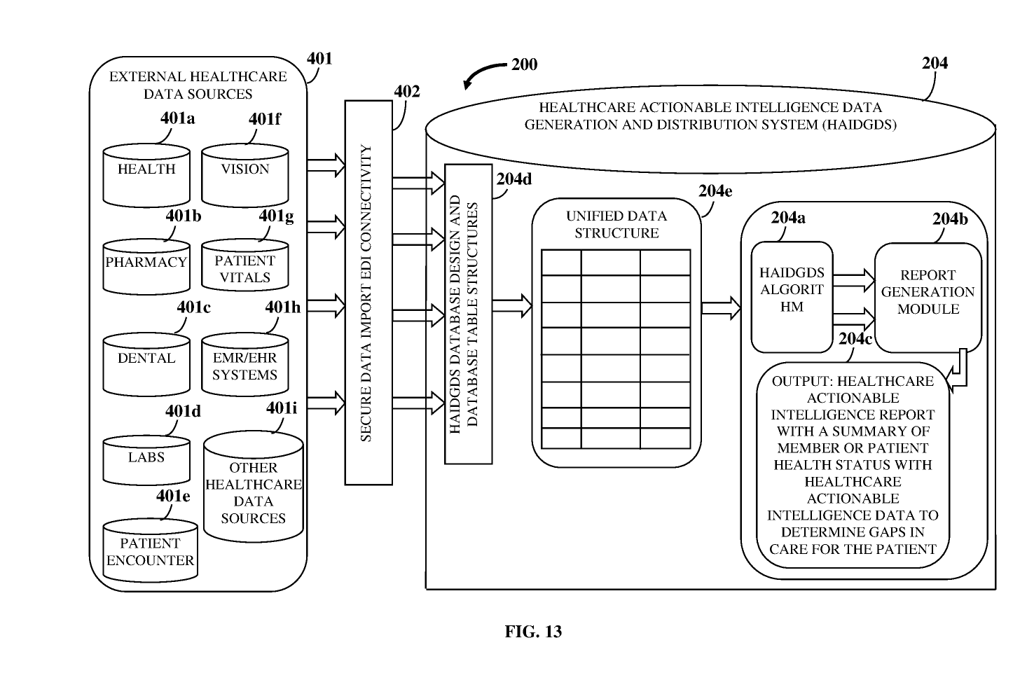

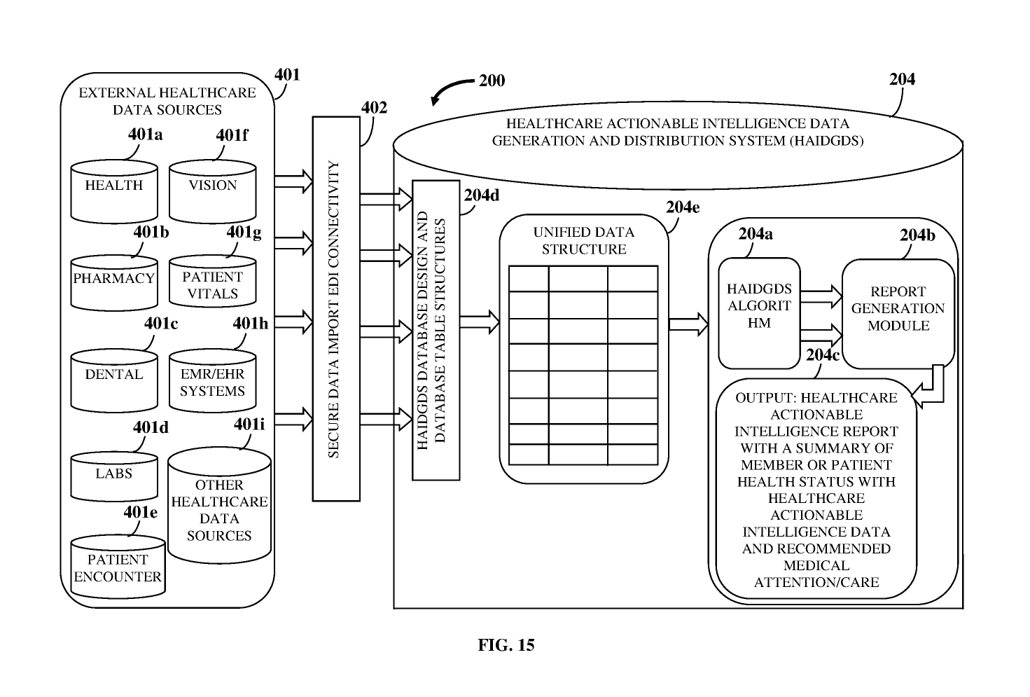

FIG. 9 exemplarily illustrates a system 200 for securely generating and distributing healthcare actionable intelligence data related to avoidable drug impacted medical costs to external third party administrator systems 901, for example, providers, plan sponsors, payers, pharmacy, etc. The healthcare actionable intelligence data generation and distribution system (HAIDGDS) 204 imports healthcare data sets of a patient from multiple healthcare data sources 401 via a secure data import electronic data interchange (EDI) connectivity mode 402 as disclosed in the detailed description of FIG. 4. The HAIDGDS 204 executes the HAIDGDS algorithm 204a and the report generation module 204b to transform the imported healthcare data sets in the unified data structure 204e exemplarily illustrated in FIG. 7, FIG. 13, and FIGS. 15-17, into the healthcare actionable intelligence report 204c, which is a comprehensive summary report of the patient's health status for a requesting entity's review and decision support as disclosed in the detailed description of FIG. 1. The HAIDGDS 204 publishes an active, clickable uniform resource identifier (URI) to the requesting entity, for example, a third party administrator (TPA) system 901 via a communication network 206, for example, the internet. A third party administrator (TPA) clicks the active, clickable URI via the TPA system 901 and views a popup window with the healthcare actionable intelligence report 204c as delivered by the HAIDGDS 204. The TPA reviews the healthcare actionable intelligence data comprising the overall patient health status, the recommendations, and the alerts among other healthcare actionable intelligence data contained in the patient specific healthcare actionable intelligence report 204c for creating an informed pharmacy drug care plan to optimally manage the patient's health and avoid drug induced medical costs. The TPA is able to avoid additional drug induced medical care needed by prescribing pharmacy drugs that are safe to use in combination with other existing prescription drugs.

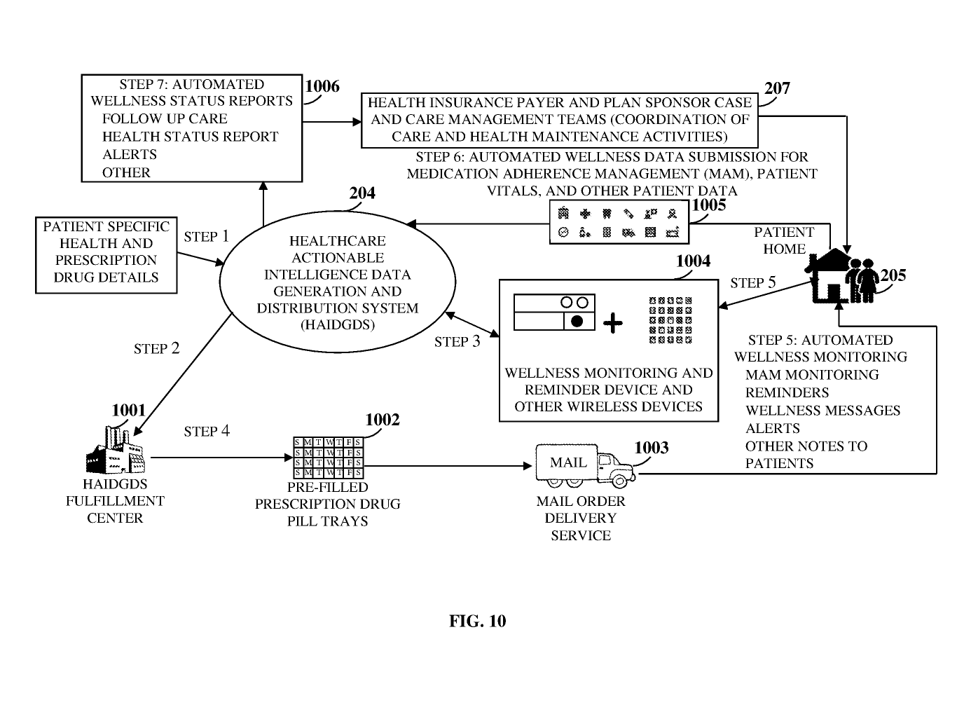

FIG. 10 exemplarily illustrates a flow diagram showing an implementation of the healthcare actionable intelligence data generation and distribution system (HAIDGDS) 204 for securely generating and distributing healthcare actionable intelligence data related to patient health and pharmacy drug utilization to a wellness monitoring and reminder device or a home health gateway device and other wireless devices 1004 of a patient 205, for a follow up related to patient health and pharmacy drug utilization. The HAIDGDS 204 provides a wellness monitoring and reminder device and other wireless devices 1004 to the patient 205 that allow the HAIDGDS 204 to communicate with the patient 205. The HAIDGDS 204 imports healthcare data sets of the patient 205 from multiple healthcare data sources 401 via a secure data import electronic data interchange (EDI) connectivity mode 402 exemplarily illustrated in FIG. 6, and also imports wellness monitoring data of the patient 205, reminders, etc., from the wellness monitoring and reminder device and other wireless devices 1004. The HAIDGDS 204 executes the HAIDGDS algorithm 204a for transforming the imported healthcare data sets in the unified data structure 204e exemplarily illustrated in FIG. 7, FIG. 13, and FIGS. 15-17, into a healthcare actionable intelligence report 204c exemplarily illustrated in FIG. 6, which is a comprehensive summary report for a requesting entity's review and decision support as disclosed in the detailed description of FIG. 1. The HAIDGDS 204 pushes a Health Insurance Portability and Accountability Act (HIPAA) compliant, active, clickable uniform resource identifier (URI) to the patient's 205 wellness monitoring and reminder device and other wireless devices 1004 via the communication network 206 exemplarily illustrated in FIG. 2 and FIG. 6, for example, the mobile cloud. The requesting entities, for example, a healthcare provider, a pharmacist, a care-case management team 207 exemplarily illustrated in FIG. 2, and the patient 205 click the active, clickable URI and view a popup window with the healthcare actionable intelligence report 204c as delivered by the HAIDGDS 204. The requesting entities review the patient specific healthcare actionable intelligence report 204c comprising the overall patient health status, the recommendations, and the alerts among other healthcare actionable intelligence data. The requesting entities determine the next steps in delivering medical care to the patient 205.

As exemplarily illustrated in FIG. 10, the healthcare actionable intelligence data generation and distribution system (HAIDGDS) 204 connects the requesting entities, for example, a healthcare provider, a pharmacist, a care-case management teams 207, and the patient 205. A requesting entity, for example, a practitioner transmits a request to place an order for a prescription drug to a pharmacy or a point of sale (POS) 203 exemplarily illustrated in FIG. 2, based on the transmitted healthcare actionable intelligence report 204c comprising, for example, patient specific health and prescription drug details, for drug delivery to the patient 205. Upon receiving the healthcare actionable intelligence report 204c from the HAIDGDS 204, the practitioner at the point of care (POC) 202 exemplarily illustrated in FIG. 2, refers the electronic medical record (EMR) or available electronic prescribing software and the healthcare actionable intelligence report 204c to transmit the patient's 205 prescription to the POS 203. A fulfillment center 1001 of the HAIDGDS 204 receives the patient's 205 prescription. The fulfillment center 1001 dispenses the prescribed medication to the patient 205 by a mail order delivery service 1003 in pre-filled prescription drug pill trays 1002. In an embodiment, the POS 203 dispenses the prescribed medication to the patient 205 at the location of the POS 203 when the patient 205 visits the POS 203 to collect the prescribed medication. The HAIDGDS 204 performs medication adherence management (MAM) monitoring and transmits reminders, wellness messages, alerts, and other notes to the patient 205. The HAIDGDS 204 receives automated wellness data submission 1005 for medication adherence management, patient vitals, and other patient data from the patient 205. The HAIDGDS 204 provides automated wellness status reports 1006 comprising, for example, follow up care, a health status report or the healthcare actionable intelligence report 204c, alerts, and other reports to a payer and plan sponsor case and care management teams 207 for coordination and health maintenance activities, and in turn to the patient 205.

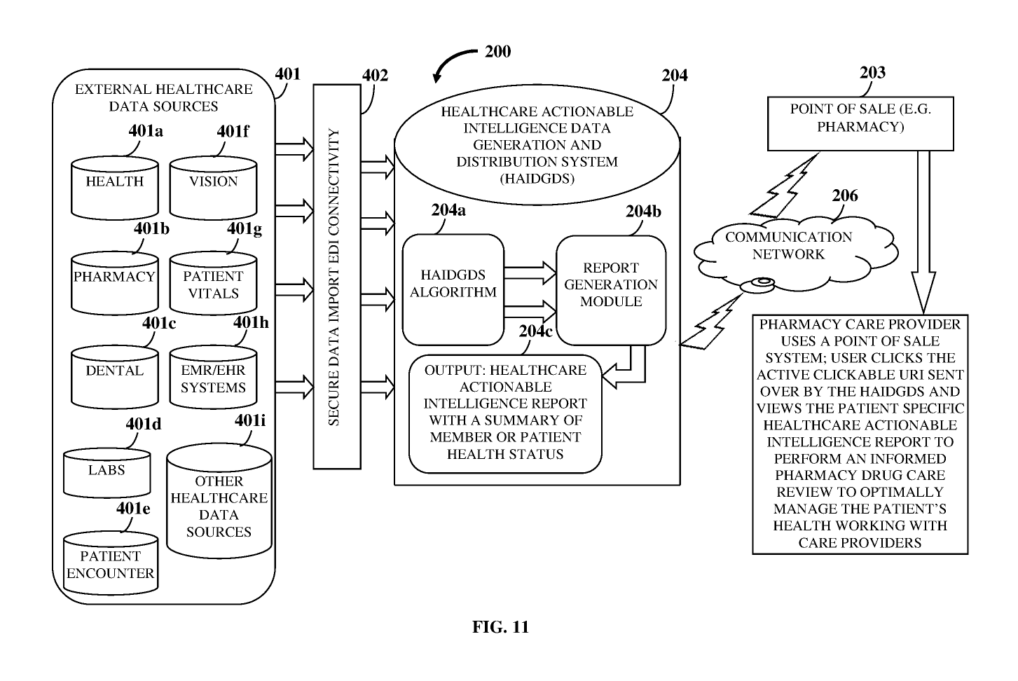

FIG. 11 exemplarily illustrates a system 200 for securely generating and distributing healthcare actionable intelligence data to a point of sale (POS) system 203 using a National Council for Prescription Drug Programs (NCPDP) D.0 standard. The healthcare actionable intelligence data generation and distribution system (HAIDGDS) 204 imports healthcare data sets of a patient from multiple healthcare data sources 401 via a secure data import electronic data interchange (EDI) connectivity mode 402 as disclosed in the detailed description of FIG. 4. The HAIDGDS 204 executes the HAIDGDS algorithm 204a and the report generation module 204b for transforming the imported healthcare data sets in the unified data structure 204e exemplarily illustrated in FIG. 7, FIG. 13, and FIGS. 15-17, into the healthcare actionable intelligence report 204c, which is a comprehensive summary report of the patient's health status for a requesting entity's review and decision support as disclosed in the detailed description of FIG. 1. The HAIDGDS 204 publishes an active, clickable uniform resource identifier (URI) to a requesting entity's system, for example, a POS system 203 such as a pharmacy care provider's system through NCPDP D.0 response custom fields via the communication network 206, for example, the internet. The pharmacy care provider or a pharmacist clicks the active, clickable URI at the POS system 203 and views a popup window with the healthcare actionable intelligence report 204c as delivered by the HAIDGDS 204. The pharmacy care provider or the pharmacist reviews the healthcare actionable intelligence data comprising the overall patient health status, the recommendations, and the alerts among other healthcare actionable intelligence data contained in the patient specific healthcare actionable intelligence report 204c to perform an informed pharmacy drug care review to optimally manage the patient's health by collaborating with healthcare providers at a point of care (POC) system 202 exemplarily illustrated in FIG. 2 and FIG. 4.

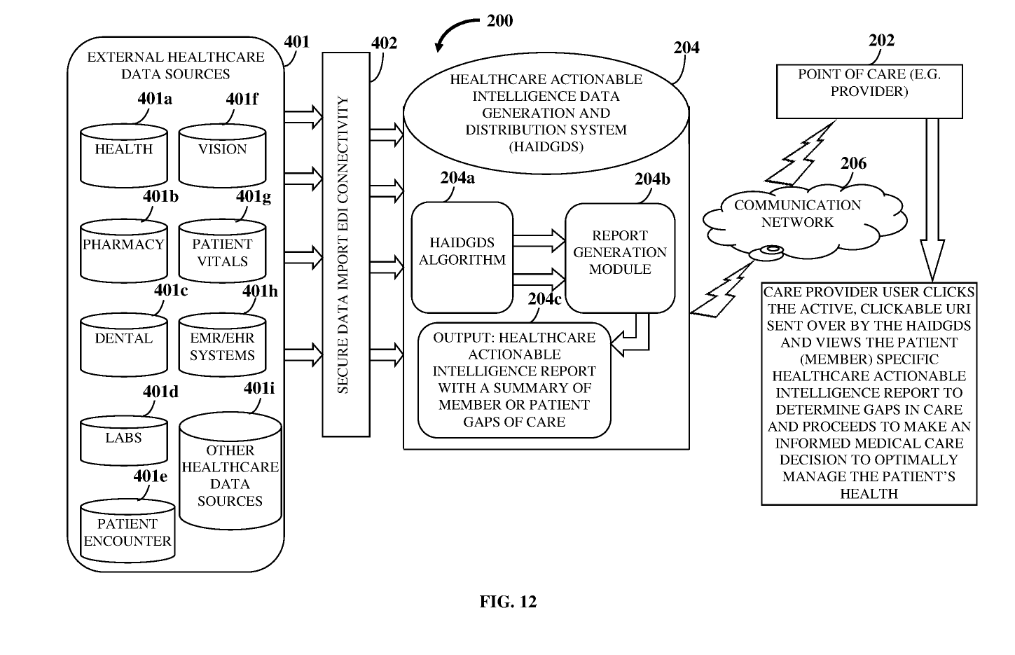

FIG. 12 exemplarily illustrates a system 200 for securely generating and distributing healthcare actionable intelligence data to a requesting entity, for example, a point of care (POC) system 202 of a requesting entity, for example, a care provider for determining gaps in care for a patient. The healthcare actionable intelligence data generation and distribution system (HAIDGDS) 204 imports healthcare data sets of a patient from multiple healthcare data sources 401 via a secure data import electronic data interchange (EDI) connectivity mode 402 as disclosed in the detailed description of FIG. 4. The HAIDGDS 204 executes the HAIDGDS algorithm 204a and the report generation module 204b for transforming the imported healthcare data sets in the unified data structure 204e exemplarily illustrated in FIG. 7, FIG. 13, and FIGS. 15-17, into the healthcare actionable intelligence report 204c, which is a comprehensive summary report of the patient's gaps of care for a requesting entity's review and decision support as disclosed in the detailed description of FIG. 1. The HAIDGDS 204 publishes an active, clickable uniform resource identifier (URI) to the care provider's POC system 202 via the communication network 206, for example, the internet. The care provider clicks the active, clickable URI at the POC system 202 and views a popup window with the patient specific healthcare actionable intelligence report 204c as delivered by the HAIDGDS 204. The care provider reviews the healthcare actionable intelligence data comprising the overall patient health status, the recommendations, and the alerts among other healthcare actionable intelligence data contained in the patient specific healthcare actionable intelligence report 204c to determine gaps in care, and proceeds to make an informed medical care decision to optimally manage the patient's health.

FIG. 13 exemplarily illustrates a system 200 for securely generating healthcare actionable intelligence data using a unified data structure 204e for determining gaps in care for a patient. The healthcare actionable intelligence data generation and distribution system (HAIDGDS) 204 imports healthcare data sets of a patient from multiple healthcare data sources 401 via a secure data import electronic data interchange (EDI) connectivity mode 402 as disclosed in the detailed description of FIG. 4. The HAIDGDS 204 implements database design and database table structures 204d and the unified data structure 204e comprising unified database data tables. The HAIDGDS 204 transforms the imported healthcare data sets into the unified data structure 204e as disclosed in the detailed description of FIG. 1. The HAIDGDS 204 analyzes the healthcare data contained in the unified data structure 204e by executing the HAIDGDS algorithm 204a to determine gaps in care for the patient and generates the healthcare actionable intelligence report 204c for the healthcare provider's review and decision support as disclosed in the detailed description of FIG. 1. The healthcare actionable intelligence report 204c comprises a summary of the patient's health status with healthcare actionable intelligence data to determine the gaps in care for the patient.

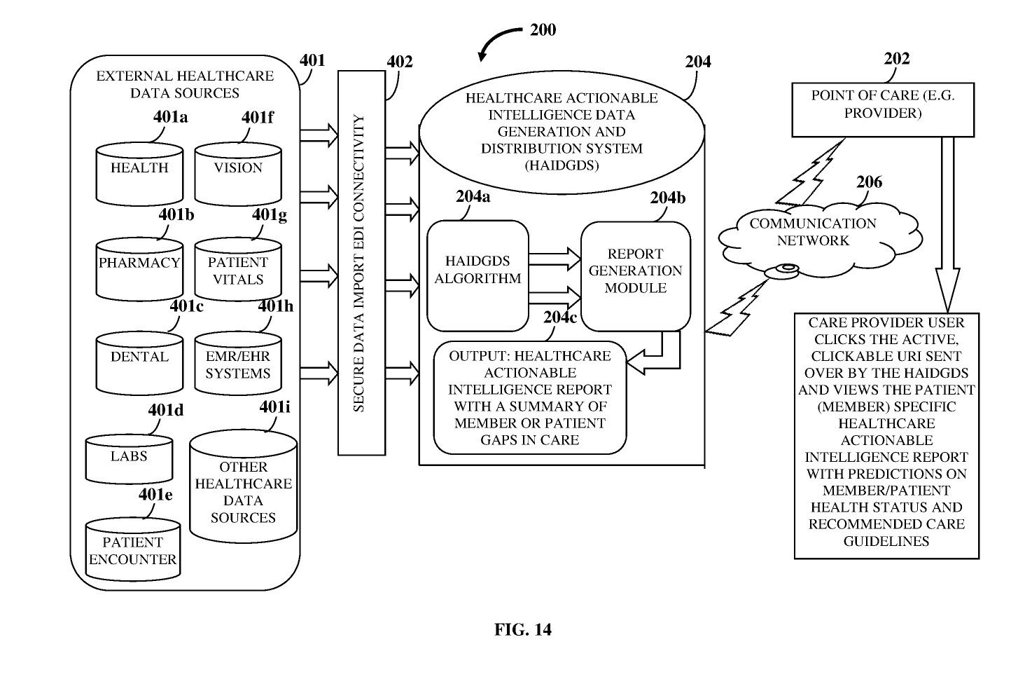

FIG. 14 exemplarily illustrates a system 200 for securely generating and distributing healthcare actionable intelligence data to a requesting entity, for example, a point of care (POC) system 202 of a care provider, for predicting patient health status and recommending appropriate medical attention and/or care to the patient. The healthcare actionable intelligence data generation and distribution system (HAIDGDS) 204 imports healthcare data sets of a patient from multiple healthcare data sources 401 via a secure data import electronic data interchange (EDI) connectivity mode 402 as disclosed in the detailed description of FIG. 4. The HAIDGDS 204 executes the HAIDGDS algorithm 204a and the report generation module 204b for transforming the imported healthcare data sets in the unified data structure 204e exemplarily illustrated in FIG. 7, FIG. 13, and FIGS. 15-17, into the healthcare actionable intelligence report 204c, which is a comprehensive summary report of the patient's gaps in care for the requesting entity's review and decision support as disclosed in the detailed description of FIG. 1. The healthcare actionable intelligence report 204c comprises predictions on the patient's health status and recommended care guidelines. The HAIDGDS 204 publishes an active, clickable uniform resource identifier (URI) to the care provider's POC system 202 via the communication network 206, for example, the internet. The care provider clicks the active, clickable URI at the POC system 202 and views a popup window with the healthcare actionable intelligence report 204c as delivered by the HAIDGDS 204. The care provider reviews the healthcare actionable intelligence data comprising the predictions on the patient health status and the recommended care guidelines contained in the patient specific healthcare actionable intelligence report 204c to make an informed medical care decision to optimally manage the patient's health.

FIG. 15 exemplarily illustrates a system 200 for securely generating healthcare actionable intelligence data using a unified data structure 204e for predicting patient health status and recommending appropriate medical attention and/or care to the patient. The healthcare actionable intelligence data generation and distribution system (HAIDGDS) 204 imports healthcare data sets of a patient from multiple healthcare data sources 401 via a secure data import electronic data interchange (EDI) connectivity mode 402 as disclosed in the detailed description of FIG. 4. The HAIDGDS 204 implements database design and table structures 204d and the unified data structure 204e comprising unified database data tables. The HAIDGDS 204 transforms the imported healthcare data sets into the unified data structure 204e as disclosed in the detailed description of FIG. 1. The HAIDGDS 204 analyzes the healthcare data contained in the unified data structure 204e by executing the HAIDGDS algorithm 204a for predicting patient health status and recommending appropriate medical attention and/or care to the patient and generates the healthcare actionable intelligence report 204c for a requesting entity's review and decision support as disclosed in the detailed description of FIG. 1. The healthcare actionable intelligence report 204c comprises a summary of the patient's health status with healthcare actionable intelligence data and recommended medical attention and/or care.

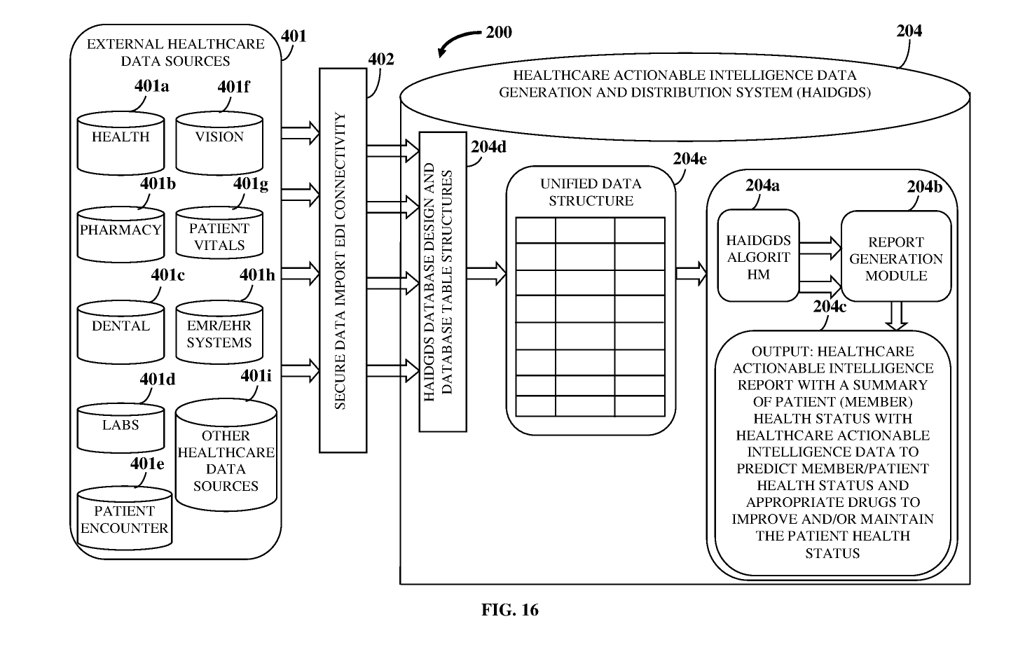

FIG. 16 exemplarily illustrates a system 200 for securely generating healthcare actionable intelligence data using a unified data structure 204e for predicting patient health status and recommending appropriate drugs to improve and/or maintain the patient health status. The healthcare actionable intelligence data generation and distribution system (HAIDGDS) 204 imports healthcare data sets of a patient from multiple healthcare data sources 401 via a secure data import electronic data interchange (EDI) connectivity mode 402 as disclosed in the detailed description of FIG. 4. The HAIDGDS 204 implements database design and table structures 204d and the unified data structure 204e comprising unified database data tables. The HAIDGDS 204 transforms the imported healthcare data sets into the unified data structure 204e as disclosed in the detailed description of FIG. 1. The HAIDGDS 204 analyzes the healthcare data contained in the unified data structure 204e by executing the HAIDGDS algorithm 204a for predicting the patient health status and recommending appropriate drugs to improve and/or maintain the patient health status and generates the healthcare actionable intelligence report 204c for a requesting entity's review and decision support as disclosed in the detailed description of FIG. 1. The healthcare actionable intelligence report 204c comprises a summary of the patient's health status with healthcare actionable intelligence data to predict the patient health status and appropriate drugs to improve and/or maintain the patient's health status.

FIG. 17 exemplarily illustrates a unified data structure 204e for storing and aggregating healthcare data sets of a patient. The unified data structure 204e comprises the transformed healthcare data sets imported from the healthcare data sources 401 exemplarily illustrated in FIGS. 4-7, FIG. 9, FIGS. 11-16, and FIG. 19. The unified data structure 204e comprises unified database data tables, for example, CALENDAR_DIM, MBR_DDIS_INTERACTION_FACT, MED_PRODUCT_DIM, MEMBER_DIM, ICD_DIM, etc., as exemplarily illustrated in FIG. 17. The CALENDAR_DIM data table stores calendar information, for example, a day, a month, a year, a date, etc., related to enrollment of a patient and scheduled visits of the patient to a point of care (POC) 202 exemplarily illustrated in FIG. 2. The CALENDAR_DIM data table comprises a primary key CALENDAR_ID_RX. The MEMBER_DIM data table comprises MEMBER_KEY as the primary key. The MEMBER_DIM data table stores information, for example, a member identifier (ID), a card ID, a health insurance claim number, a member address, a member name, a member phone number, etc., of the patient. The MED_PRODUCT_DIM data table comprises a primary key MED_PRODUCT_DIM KEY. The MED_PRODUCT_DIM data table stores information related to drugs such as national drug codes (NDC), drug descriptions, etc. The ICD_DIM data table comprises ICD_KEY as the primary key. The ICD_DIM data table stores information related to International Classification of Diseases (ICD). The MBR_DDIS_INTERACTION_FACT data table comprises MBR_DDIS_INTERACTION_KEY as a primary key and the ICD_KEY, the MEMBER_KEY, etc., as the foreign keys. The MBR_DDIS_INTERACTION_FACT data table stores information related to drug to drug interactions.

Consider an example where a patient enrolls into a health insurance plan. The healthcare actionable intelligence data generation and distribution system (HAIDGDS) 204 reads the healthcare eligibility request received from a requesting entity, for example, an insurance company, identifies the patient from the healthcare eligibility request, and retrieves and compiles healthcare data sets of the identified patient from multiple healthcare data sources 401 via a secure data import electronic connectivity mode 402 as disclosed in the detailed description of FIG. 1. The HAIDGDS 204 generates and processes insurance claims data in the form of healthcare data sets for one or more clinical services, for example, a medical service, a vision service, a dental service, a laboratory service, a pharmaceutical service, etc., utilized by the identified patient. The HAIDGDS 204 extracts and stores information, for example, patient information, diagnosis information, information on medical procedures, medication information, laboratory information, visit information, etc., to the patient's profile that is identified by a unique identifier and stored in the unified data structure 204e. For example, the HAIDGDS 204 stores the healthcare data sets comprising, for example, patient information into the MEMBER_DIM data table of the unified data structure 204e, visit information into the CALENDAR_DIM data table of the unified data structure 204e, medication information into the MED_PRODUCT_DIM data table of the unified data structure 204e, the International Classification of Diseases (ICD) information into the ICD_DIM data table of the unified data structure 204e, and drug to drug interaction information into the MBR_DDIS_INTERACTION_FACT data table of the unified data structure 204e. The HAIDGDS 204 analyzes the healthcare data stored in the unified data structure 204e by executing the HAIDGDS algorithm 204a exemplarily illustrated in FIGS. 4-7, FIG. 9, and FIGS. 11-16, to identify healthcare actionable intelligence data related to the patient's health and to generate alerts such as therapeutic duplication, drug interactions, adverse drug events, high risk alerts, gaps in care, continuity of care, medication optimization, etc., as disclosed in the detailed description of FIG. 1.

FIG. 18 exemplarily illustrates a screenshot of a healthcare actionable intelligence report 204c generated by the healthcare actionable intelligence data generation and distribution system (HAIDGDS) 204 exemplarily illustrated in FIG. 2. The healthcare actionable intelligence report 204c comprises patient information 1801, social history 1802, current complaints 1803 of the patient, information related to vitals 1804 of the patient, visit history 1805 of the patient, current clinical status 1806 of the patient, and past clinical status 1807 of the patient as exemplarily illustrated in FIG. 18. The patient information 1801 comprises, for example, name, age, gender, etc., of the patient, name and contact number of a primary care physician of the patient, etc. The social history 1802 comprises, for example, marital status, living situation, nutritional status, etc. The current complaints 1803 comprise, for example, headache since afternoon, dizziness since morning, etc. The information related to the vitals 1804 in the healthcare actionable intelligence report 204c comprises, for example, temperature, weight, height, blood pressure level, heart rate, body mass index, glucose level, etc., of the patient. The visit history 1805 comprises, for example, number of emergency room visits, inpatient visits, outpatient visits, etc. The HAIDGDS 204 highlights the values that are above a normal prescribed level in the healthcare actionable intelligence report 204c, thereby indicating risk factors in the healthcare actionable intelligence report 204c. The HAIDGDS 204 statistically analyzes the healthcare data sets contained in the unified data structure 204e exemplarily illustrated in FIG. 17, to derive the overall patient health status of the patient. The HAIDGDS algorithm 204a exemplarily illustrated in FIGS. 4-7, FIG. 9, and FIGS. 11-16, leverages, for example, the patient information 1801, number of emergency room visits, number of medications, duration of stay in a hospital, severity of illness, identified care gaps, and number of comorbid conditions among other relevant information from the unified data structure 204e to generate a normalized severity score that lies between 0 and 100, with 0 being the lowest severity score and 100 being the highest severity score within the healthcare data sets contained in the unified data structure 204e. In an embodiment, the HAIDGDS 204 frequently updates the normalized severity score using a home based vital measurements score and medication adherence scores. The HAIDGDS 204 utilizes the updated normalized severity score to indicate the severity of care required by the patient and appropriateness of a current regimen to a point of care (POC) 202 exemplarily illustrated in FIG. 2.

Consider an example where a patient is diagnosed with coronary artery disease, underwent a percutaneous transluminal coronary angioplasty (PTCA), and is prescribed a drug Prasugrel which is a platelet aggregation inhibitor, post procedure. When a prescription for the drug is generated, on identifying the patient using a member identifier from a healthcare eligibility request received from a requesting entity, for example, at a point of care (POC) 202 or a point of sale (POS) 203 exemplarily illustrated in FIG. 2, the healthcare actionable intelligence data generation and distribution system (HAIDGDS) 204 retrieves and compiles the healthcare data sets of the identified patient from multiple healthcare data sources 401, and stores and transforms the retrieved and compiled healthcare data sets into a unified data structure 204e. The HAIDGDS 204 analyzes healthcare data contained in the unified data structure 204e and identifies a drug Plavix already prescribed to the patient prior to the PTCA procedure by a primary care physician. The drug Plavix is another drug from the same therapeutic class as the drug Prasugrel. The HAIDGDS 204 determines overall patient health status and generates healthcare recommendations and alerts for the identified patient by analyzing the healthcare data contained in the unified data structure 204e. The HAIDGDS 204 analyzes the Plavix drug request with the existing medication list. The HAIDGDS 204 executes the HAIDGDS algorithm 204a and determines the Plavix drug request is an event of therapeutic duplication that is dangerous to the patient's health and highlights the entry in the healthcare actionable intelligence report 204c as exemplarily illustrated in FIG. 18. The healthcare actionable intelligence report 204c indicates the medical condition, that is, coronary artery disease 1808, the existing medication, that is, Prasugrel 1809, the new prescription, that is, Plavix 1810, the occurrence of therapeutic duplication 1811, and high dose and risk alerts 1812 in the healthcare actionable intelligence report 204c as exemplarily illustrated in FIG. 18. The HAIDGDS 204 transmits an alert on the therapeutic duplication 1811 via a communication channel of the communication network 206 exemplarily illustrated in FIG. 2, to the point of care 202 or the point of sale 203, to allow a healthcare provider at the point of care 202 or the point of sale 203 to take appropriate action to prevent a dangerous situation.

Consider another example where the healthcare actionable intelligence data generation and distribution system (HAIDGDS) 204 receives a healthcare eligibility request from a requesting entity, for example, a primary care physician. The HAIDGDS 204 identifies the patient from the received healthcare eligibility request. The HAIDGDS 204 retrieves and compiles the healthcare data sets of the identified patient from multiple healthcare data sources 401. The HAIDGDS 204 stores and transforms the retrieved and compiled healthcare data sets into a unified data structure 204e. On analyzing the healthcare data contained in the unified data structure 204e, the HAIDGDS 204 generates and transmits healthcare actionable intelligence data to a user device of the primary care physician via the healthcare actionable intelligence report 204c. In this example, the healthcare actionable intelligence data informs the primary care physician that the patient is currently prescribed six branded drugs for which the patient's out of pocket expense is $220 and that the equivalent food and drug administration (FDA) approved generics are available for which the out of pocket expense would be only $60. The primary care physician, upon receiving and reviewing this healthcare actionable intelligence data from the healthcare actionable intelligence report 204c generated by the HAIDGDS 204, optimizes a medication list of the patient to reduce the economic burden on the patient.

FIG. 19 exemplarily illustrates the system 200 comprising the healthcare actionable intelligence data generation and distribution system (HAIDGDS) 204 for securely generating and distributing healthcare actionable intelligence data to requesting entities in a computing environment. The HAIDGDS 204 is a computer system that is programmable using a high level computer programming language. In an embodiment, the HAIDGDS 204 is implemented on a computing device, for example, one or more of a personal computer, a tablet computing device, a mobile computer, a smartphone, a portable computing device, a laptop, a touch centric device, a workstation, a server, a portable electronic device, a network enabled computing device, an interactive network enabled communication device, any other suitable computing equipment, combinations of multiple pieces of computing equipment, etc., using programmed and purposeful hardware. In an embodiment, the HAIDGDS 204 is configured as a cloud computing based platform implemented as a service. For example, the HAIDGDS 204 is configured as a software as a service (SaaS) platform or a platform as a service (PaaS) that securely generates and distributes healthcare actionable intelligence data to requesting entities.

The healthcare actionable intelligence data generation and distribution system (HAIDGDS) 204 communicates with multiple healthcare data sources 401 via a secure data import electronic data interchange (EDI) connectivity mode 402. The HAIDGDS 204 also communicates with a point of care 202, a point of sale 203, a member or a patient 205 exemplarily illustrated in FIG. 2, via a user device 1911, and a third party administrator system 901 via a communication network 206, for example, a short range network or a long range network. The HAIDGDS 204 interfaces with the health data sources 401, the point of care 202, the point of sale 203, the user device 1911, and the third party administrator system 901 for securely generating and distributing healthcare actionable intelligence data to requesting entities in a computing environment, and therefore more than one specifically programmed computing system is used for implementing healthcare actionable intelligence data generation and processing of the method disclosed herein in a healthcare environment. The user device 1911 is an electronic device, for example, one or more of a personal computer, a tablet computing device, a mobile computer, a mobile phone, a smartphone, a portable computing device, a personal digital assistant, a laptop, a wearable computing device such as the Google Glass® of Google Inc., the Apple Watch® of Apple Inc., the Android Smartwatch® of Google Inc., etc., a touch centric device, a client device, a portable electronic device, a network enabled computing device, an interactive network enabled communication device, a gaming device, an image capture device, any other suitable computing equipment, combinations of multiple pieces of computing equipment, etc. In an embodiment, the user device 1911 is a hybrid computing device that combines the functionality of multiple devices. Examples of a hybrid computing device comprise a cellular telephone that includes a media player functionality, a tablet device that includes a wireless communications capability, a cellular telephone that includes game and multimedia functions, and a portable device that receives electronic mail (email), supports mobile telephone calls, has a media player functionality, and supports web browsing. In an embodiment, computing equipment is used to implement applications such as media playback applications, a web browser, an electronic mail (email) application, a calendar application, etc.

The communication network 206 is, for example, one of the internet, an intranet, a wired network, a wireless network, a network that implements Bluetooth® of Bluetooth Sig, Inc., a network that implements Wi-Fi® of Wi-Fi Alliance Corporation, an ultra-wideband communication network (UWB), a wireless universal serial bus (USB) communication network, a communication network that implements ZigBee® of ZigBee Alliance Corporation, a general packet radio service (GPRS) network, a mobile telecommunication network such as a global system for mobile (GSM) communications network, a code division multiple access (CDMA) network, a third generation (3G) mobile communication network, a fourth generation (4G) mobile communication network, a fifth generation (5G) mobile communication network, a long-term evolution (LTE) mobile communication network, a public telephone network, etc., a local area network, a wide area network, an internet connection network, an infrared communication network, etc., or a network formed from any combination of these networks. In an embodiment, the healthcare actionable intelligence data generation and distribution system (HAIDGDS) 204 is accessible to users, for example, through a broad spectrum of technologies and devices such as personal computers with access to the internet, internet enabled cellular phones, tablet computing devices, etc.

As exemplarily illustrated in FIG. 19, the healthcare actionable intelligence data generation and distribution system (HAIDGDS) 204 comprises a non-transitory computer readable storage medium, for example, a memory unit 1903 for storing programs and data, and at least one processor 1901 communicatively coupled to the non-transitory computer readable storage medium. As used herein, “non-transitory computer readable storage medium” refers to all computer readable media, for example, non-volatile media, volatile media, and transmission media, except for a transitory, propagating signal. Non-volatile media comprise, for example, solid state drives, optical discs or magnetic disks, and other persistent memory volatile media including a dynamic random access memory (DRAM), which typically constitute a main memory. Volatile media comprise, for example, a register memory, a processor cache, a random access memory (RAM), etc. Transmission media comprise, for example, coaxial cables, copper wire, fiber optic cables, modems, etc., including wires that constitute a system bus coupled to the processor 1901. The non-transitory computer readable storage medium is configured to store computer program instructions defined by modules, for example, 1903a, 1903b, 1903c, 1903d, 1903e, 204b, 1903f, 1903g, etc., of the HAIDGDS 204. The modules, for example, 1903a, 1903b, 1903c, 1903d, 1903e, 204b, 1903f, 1903g, etc., of the HAIDGDS 204 are installed and stored in the memory unit 1903 of the HAIDGDS 204. The memory unit 1903 is used for storing program instructions, applications, and data. The memory unit 1903 is, for example, a random access memory (RAM) or another type of dynamic storage device that stores information and instructions for execution by the processor 1901. The memory unit 1903 also stores temporary variables and other intermediate information used during execution of the instructions by the processor 1901. The HAIDGDS 204 further comprises a read only memory (ROM) or another type of static storage device that stores static information and instructions for the processor 1901.

The processor 1901 of the healthcare actionable intelligence data generation and distribution system (HAIDGDS) 204 is configured to execute the computer program instructions defined by the modules, for example, 1903a, 1903b, 1903c, 1903d, 1903e, 204b, 1903f, 1903g, etc., of the HAIDGDS 204. The processor 1901 refers to any of one or more microprocessors, central processing unit (CPU) devices, finite state machines, computers, microcontrollers, digital signal processors, logic, a logic device, a user circuit, an application specific integrated circuit (ASIC), a field-programmable gate array (FPGA), a chip, etc., or any combination thereof, capable of executing computer programs or a series of commands, instructions, or state transitions. In an embodiment, the processor 1901 is implemented as a processor set comprising, for example, a programmed microprocessor and a math or graphics co-processor. The processor 1901 is selected, for example, from the Intel® processors such as the Itanium® microprocessor or the Pentium® processors, Advanced Micro Devices (AMD®) processors such as the Athlon® processor, UltraSPARC® processors, microSPARC® processors, HP® processors, International Business Machines (IBM®) processors such as the PowerPC® microprocessor, the MIPS® reduced instruction set computer (RISC) processor of MIPS Technologies, Inc., RISC based computer processors of ARM Holdings, Motorola® processors, Qualcomm® processors, etc. The HAIDGDS 204 disclosed herein is not limited to employing a processor 1901. In an embodiment, the HAIDGDS 204 employs a controller or a microcontroller. The processor 1901 executes the modules, for example, 1903a, 1903b, 1903c, 1903d, 1903e, 204b, 1903f, 1903g, etc., of the HAIDGDS 204.

As exemplarily illustrated in FIG. 19, the healthcare actionable intelligence data generation and distribution system (HAIDGDS) 204 further comprises a display unit 1902, a data bus 1904, a network interface 1905, an input/output (I/O) controller 1906, input devices 1907, a fixed media drive 1908 such as a hard drive, a removable media drive 1909 for receiving removable media, output devices 1910, etc. The display unit 1902, via the graphical user interface (GUI) 1902a, displays information, display interfaces, etc., for example, for displaying a healthcare eligibility request received from one or more requesting entities, displaying the healthcare actionable intelligence report 204c comprising healthcare actionable intelligence data exemplarily illustrated in FIGS. 4-7, FIG. 9, FIGS. 11-16, and FIG. 18, etc. The display unit 1902, via the GUI 1902a, also displays user interface elements such as input fields, buttons, swipable arrows, icons, etc., for accessing the healthcare eligibility request, the healthcare actionable intelligence report 204c, etc.

The display unit 1902 comprises, for example, a video display, a liquid crystal display, a plasma display, an organic light emitting diode (OLED) based display, etc. The healthcare actionable intelligence data generation and distribution system (HAIDGDS) 204 renders the graphical user interface (GUI) 1902a on the display unit 1902 to receive user inputs, the healthcare eligibility request, etc. The display unit 1902 displays the GUI 1902a. The GUI 1902a is, for example, an online web interface, a web based downloadable application interface, a mobile based downloadable application interface, etc. In an embodiment, the GUI 1902a allows a user of the HAIDGDS 204 to input, for example, a healthcare eligibility request into the HAIDGDS 204. The input devices 1907 are used for inputting data, for example, medical claim data, laboratory reports, etc., into the HAIDGDS 204 and for routine maintenance of the HAIDGDS 204. In an embodiment, the user uses the input devices 1907 to provide inputs, for example, the healthcare eligibility request to the HAIDGDS 204. The input devices 1907 are, for example, a keyboard such as an alphanumeric keyboard, a microphone, a joystick, a pointing device such as a computer mouse, a touch pad, a light pen, a physical button, a touch sensitive display device, a track ball, a pointing stick, any device capable of sensing a tactile input, etc. The output devices 1910 output the results of operations performed by the HAIDGDS 204. For example, in an embodiment, the HAIDGDS 204 renders the generated healthcare actionable intelligence report 204c to the user of the HAIDGDS 204 using the output devices 1910.

The data bus 1904 permits communications between the modules, for example, 1903a, 1903b, 1903c, 1903d, 1903e, 204b, 1903f, 1903g, 1903h, etc., of the healthcare actionable intelligence data generation and distribution system (HAIDGDS) 204. The network interface 1905 enables connection of the HAIDGDS 204 to the communication network 206. In an embodiment, the network interface 1905 is provided as an interface card also referred to as a “line card”. The network interface 1905 comprises, for example, one or more of an infrared (IR) interface, an interface implementing Wi-Fi® of Wi-Fi Alliance Corporation, a universal serial bus (USB) interface, a FireWire® interface of Apple Inc., an Ethernet interface, a frame relay interface, a cable interface, a digital subscriber line interface, a token ring interface, a peripheral controller interconnect interface, a local area network interface, a wide area network interface, interfaces using serial protocols, interfaces using parallel protocols, Ethernet communication interfaces, asynchronous transfer mode interfaces, a high speed serial interface, a fiber distributed data interface, interfaces based on a transmission control protocol/internet protocol, interfaces based on wireless communications technology such as satellite technology, radio frequency technology, near field communication, etc. The I/O controller 1906 controls input actions and output actions performed by the HAIDGDS 204.

The modules of the healthcare actionable intelligence data generation and distribution system (HAIDGDS) 204 comprise a data reception module 1903a, a patient identification module 1903b, a healthcare data compiler 1903c, a healthcare data storage and transformation module 1903d, a healthcare data analysis module 1903e, a report generation module 204b, a messaging module 1903f, a masking module 1903g, and a database 1903h. The data reception module 1903a receives a healthcare eligibility request from multiple requesting entities. The patient identification module 1903b identifies a patient 205 from the received healthcare eligibility request, for example, by reading demographic information of a patient 205 from data fields of the received healthcare eligibility request. An example of a code snippet of the patient identification module 1903b executed by the processor 1901 of the HAIDGDS 204 for identifying the patient 205 from the received healthcare eligibility request is disclosed below:

```
private readonly UnitOfWork unitOfWork = new UnitOfWork( );
public IQueryable<EligibilityHeaderVM> GetEligibilityHeaders(decimal? pd_plan_id)
{
  IQueryable<EligibilityHeaderVM> criterialist = null;
  if (pd_plan_id != null)
  {
    criterialist = (from statuslst in unitOfWork.VW_PBP_WF_STATUS_
    MAX_MAINRepository.Get( )
        join pbpElig in
        unitOfWork.PBP_ELIGIBIITY_HEADERRepository.Get( ).AsEnu
        merable( ) on statuslst.PBP_ELIGIBILITY_ID_RX equals
        pbpElig.PBP_ELIGIBILITY_ID_RX
        where statuslst.PBP_ID_RX = = pd_plan_id &&
        Convert.ToDateTime
        (pbpElig.ELG_START_DT).Year>=DateTime.Today.Year
        select new EligibilityHeaderVM
        {
          PbpEligibility_Id_Rx =
          statuslst.PBP_ELIGIBILITY_ID_RX,
          PBP_ELIGIBILITY_NAME = pbpElig.
          PBP_ELIGIBILITY_NAME,
          Pbp_Id_Rx = statuslst.PBP_ID_RX,
          ElgStartDate = statuslst.ELG_START_DT,
          ElgEndDate = statuslst.ELG_END_DT,
          Status = statuslst.WF_STATUS_CD,
          WFStatusID = statuslst.WF_STATUS_ID_RX,
          WFOrderID = statuslst.WF_ORDER_ID
        });
  }
  else
  {
      criterialist = (from statuslst in
      unitOfWork.VW_PBP_WF_STATUS_MAX_MAINRepository.Get( )
      join pbpElig in
      unitOfWork.PBP_ELIGIBIITY_HEADERRepository.Get( ).AsEnumerabl
      e( ) on statuslst.PBP ELIGIBILITY_ID_RX equals
      pbpElig.PBP_ELIGIBILITY_ID_RX
      where pbpElig.ELG_START_DT.Value.Year >= DateTime.Today.Year
      select new EligibilityHeaderVM
      {
        PbpEligibility_Id_Rx = statuslst.PBP_ELIGIBILITY_ID_RX,
        Pbp_Id_Rx = statuslst.PBP_ID_RX,
        PBP_ELIGIBILITY_NAME =
        pbpElig.PBP_ELIGIBILITY_NAME,
        ElgStartDate = statuslst.ELG_START_DT,
        ElgEndDate = statuslst.ELG_END_DT,
        Status = statuslst.WF_STATUS_CD,
        WFStatusID = statuslst.WF_STATUS_ID_RX,
        WFOrderID = statuslst.WF_ORDER_ID
      });
  }
  return criterialist;
}
```

The healthcare data compiler 1903c retrieves and compiles healthcare data sets of the identified patient 205 from one or more external data sources, for example, the healthcare data sources 401 comprising precompiled data sources via the secure data import electronic connectivity mode 402. An example of a code snippet of the healthcare data compiler 1903c executed by the processor 1901 of the healthcare actionable intelligence data generation and distribution system (HAIDGDS) 204 for retrieving and compiling the healthcare data sets of the identified patient 205 from the healthcare data sources 401 via the secure data import electronic connectivity mode 402 is disclosed below:

```
public RequestParserOutput DOX12TOXML(string x12Data)
{
  _logger = TempLogger.LoggerInstance;
  _HeaderCollection = new List<X12RequestDataHolder>( );
  _DetailCollection = new List<X12RequestDataHolder>( );
  _TrailerCollection = new List<X12RequestDataHolder>( );
  // Fill Collections
  string[ ] segmentCollection = x12Data.Split(new char[ ] {
  SEGMENT_SEPERATOR }, StringSplitOptions.RemoveEmptyEntries);
  string[ ] dataElementCollection = segmentCollection[2].Split(new char[ ]
  {DATA_ELEMENT_SEPERATOR }, StringSplitOptions.RemoveEmptyEntries);
  // Call Validator
  _logger.AddLogMessage(“Calling Request EDI Validator :” +
  Environment.NewLine);
  I270Validator EDI270Validator = new EDI270Validator(x12Data);
  string validationResult = EDI270Validator.Validate270EDI( );
  if (validationResult != “VALID270”)
  {
    _logger.AddLogMessage(“999 generated, returning back to caller, no further
    processing to be done :” + Environment.NewLine);
    // 999 is generated
    requestParserOutput.EDI999_Response_to_270 = validationResult;
    requestParserOutput.Is999Raised = true;
    return requestParserOutput;
  }
  else
  {
    _logger.AddLogMessage(“Request EDI Validation Complete :” +
    Environment.NewLine);
    //HS (270) Eligibility, Coverage or Benefit Inquiry
    // FA Implementation Acknowledgement (999)
    _TransactionType = dataElementCollection[1];
    if (_TransactionType == X12TransactionTypes.X12270)
    {
      string[ ] Components = x12Data.Trim( ).Split(new[ ]
    {COMPONENT_SEPERATOR }, StringSplitOptions.RemoveEmptyEntries);
      x12Data = Components [0] + Components [1];
    }
  }
}
```

The healthcare data storage and transformation module 1903d stores the retrieved and compiled healthcare data sets into data tables of the database 1903h and transforms the stored healthcare data sets into a unified data structure 204e as exemplarily illustrated in FIG. 7, FIG. 13, and FIGS. 15-17. An example of a code snippet of the healthcare data storage and transformation module 1903d executed by the processor 1901 of the healthcare actionable intelligence data generation and distribution system (HAIDGDS) 204 for storing and transforming the retrieved and compiled healthcare data sets into the unified data structure 204e is disclosed below:

```
PROCEDURE T270_XML_LOAD(
    p_xml IN XMLType,
    p_t270_hdr_id_rx OUT NUMBER,
    CUR_RESPONSE OUT SYS_REFCURSOR) AS
/*********************************************************************
```

| | |
|---|---|
| NAME: | T270_XML_LOAD |
| PURPOSE: | Load XML 270 data (request) into 270 DB structure |
| PARMS: | p_xml (270 Eligibility Benefit Request transaction XML) |
| RETURN: | p_t270_hdr_id_rx (270 Eligibility Benefit Response transaction primary |

```
    key ) cursor CUR RESPONSE (support response results, including errors)
/*********************************************************************/
v_start_time_timestamp := systimestamp;
v_info_src_dtl_id_rx_number;
v_subscr_dtl_id_rx_number;
BEGIN
  dbms_output.enable(null);
  dbms_output.put_line(‘Start 270 load Time: ’ || to_char(SYSTIMESTAMP, ‘DD-
    MM-YYYY HH:MI:SS:FF’));
  --generate header id
  p_t270_hdr_id_rx := T270_HEADER_SEQ.NEXTVAL;
  --insert log 270 XML
  LOG_PRC(p_t270_hdr_id_rx, ‘270XML’, null, null, null, p_xml);
  --load header data
  FOR t270 IN (
    SELECT isa_inf_qlf, isa_inf, isa_sec_inf_qlf, trim(isa_sec_inf) isa_sec_inf,
    isa_intr_send_id_qlf, trim(isa_intr_send_id) isa_intr_send_id, isa_intr_rcv_id_qlf,
    trim(isa_intr_rcv_id) isa_intr_rcv_id, isa_intr_dt, isa_intr_tm, isa_intr_rep_sep,
    isa_intr_ctrl_ver, isa_intr_ctrl_num, isa_ack_cd, isa_use_ind, isa_comp_elm_sep,
    gs_id, gs_app_sender_cd, gs_app_rcv_cd, gs_dt, gs_tm, gs_ctrl_num,
    gs_agency_cd, gs_vers_id, st_id, st_ctrl_num, st_imp_ref, bht_hstr_cd,
    bht_tran_set_cd, bht_ref_id, bht_dt, bht_tm, se_num, -- trailer se_ctrl_num,
    ge_num, ge_ctrl_num, iea_num, iea_ctrl_num
    FROM
      xmltable(‘Transmission’ passing p_xml
        COLUMNS
    isa_inf_qlf VARCHAR2 (2 BYTE)   path
    ‘Header/InterchangeControlHeader/@AuthorizationInformationQualifier’,
    isa_inf   VARCHAR2 (10 BYTE) path
    ‘Header/InterchangeControlHeader/@AuthorizationInformation’,
    isa_sec_inf_qlf  VARCHAR2 (2 BYTE)   path
    ‘Header/InterchangeControlHeader/@SecurityInformationQualifier’,
    isa_sec_inf VARCHAR2 (10 BYTE)   path
    ‘Header/InterchangeControlHeader/@SecurityInformation’,
    isa_intr_send_id_qlf   VARCHAR2 (2 BYTE)   path
    ‘Header/InterchangeControlHeader/@InterchangeIDQualifier’,
    isa_intr_send_id VARCHAR2 (15 BYTE)   path
    ‘Header/InterchangeControlHeader/@InterchangeSenderID’,
Loop
--dbms_output.put_line(‘t270h.gs_dt:’||t270h.gs_dt);
--dbms_output.put_line(‘t270h.bht dt:’||t270h.bht_dt);
--insert header
INSERT INTO T270_HEADER
  ( T270_HDR_ID_RX, ISA_INF_QLF, ISA_INF, ISA_SEC_INF_QLF,
  ISA_SEC_INF, ISA_INTR_SEND_ID_QLF, ISA_INTR_SEND_ID,
  ISA_INTR_RCV_ID_QLF, ISA_INTR_RCV_ID, ISA_INTR_DT, ISA_INTR_TM,
  ISA_INTR_REP_SEP, ISA_INTR_CTRL_VER, ISA_INTR_CNTRL_NUM,
  ISA_ACK_CD, ISA_USE_IND, isa_comp_elm_sep, GS_ID,
  GS_APP_SENDER_CD, GS_APP_RCD_CD, GS_DT, GS_TM, GS_CTRL_NUM,
  GS_AGENCY_CD, GS_VERS_ID, ST_ID, ST_CTRL_NUM, ST_IMP_REF,
  BHT_HSTR_CD, BHT_TRAN_SET_CD, BHT_REF_ID, BHT_DT, BHT_TM,
  CREATED_BY, CREATED_DT
  )
  VALUES
  (
    p_t270_hdr_id_rx, t270h.isa_inf_qlf, t270h.isa_inf, t270h.isa_sec_inf_qlf,
    t270h.isa_sec_inf, t270h.isa_intr_send_id_qlf, t270h.isa_intr_send_id,
    t270h.isa_intr_rcv_id_qlf, t270h.isa_intr_rcv_id, to_date(t270h.isa_intr_dt,
    ‘RRMMDD’), t270h.isa_intr_tm, t270h.isa_intr_rep_sep, t270h.isa_intr_ctrl_ver,
    t270h.isa_intr_ctrl_num, t270h.isa_ack_cd, t270h.isa_use_ind,
    t270h.isa_comp_elm_sep, t270h.gs_id, t270h.gs_app_sender_cd,
    t270h.gs_app_rcv_cd, to_date(t270h.gs_dt, ‘RRRRMMDD’), t270h.gs_tm,
    t270h.gs_ctrl_num, t270h.gs_agency_cd, t270h.gs_vers_id, t270h.st.id,
    t270h.st_ctrl_num, t270h.st_imp_ref, t270h.bht_hstr_cd, t270.bht_tran_set_cd,
    t270h.bht_ref_id, to_date(t270h.bht_dt, ‘RRRMMDD’), t270h.bht_tm, 0,
    SYSTIMESTAMP );
INSERT INTO t270_trail
( T270_HDR_ID_RX, SE_NUM, SE_CTRL_NUM, GE_NUM, GE_CTRL_NUM,
IEA_NUM, IEA_CTRL_NUM )
VALUES
( p_t270_hdr_id_rx, t270h.se_num, t270h.se_ctrl_num, t270h.ge_num,
t270h.ge_ctrl_num, t270h.iea_num, t270h.iea_ctrl_num
);
--start detail XML loop
FOR t270dxml IN (
  SELECT records
    FROM xmltable (‘for $i in Transmission/Detail return $i’ passing p_xml
    COLUMNS records XMLTYPE path ‘/’)
    )
loop
--dbms_output.put_line(‘Det Record: ’ || t270dxml.records.getStringVal( ) );
--load detail data
FOR t270d IN (
  --2000A, 2100A Loop source
  --2000B, 2100B Loop receiver
  SELECT HL_ID,
  HL_LVL_CD,
  HL_CHLD_CD,
      ENTITY_ID,
...)
```

The healthcare data analysis module 1903e determines overall patient health status and generates healthcare recommendations and alerts for the identified patient 205 by analyzing healthcare data contained in the unified data structure 204e. The healthcare actionable intelligence data generation and distribution system (HAIDGDS) algorithm 204a exemplarily illustrated in FIGS. 4-7, FIG. 9, and FIGS. 11-16, defines the healthcare data analysis module 1903e that communicates with the report generation module 204b. The report generation module 204b generates a healthcare actionable intelligence report 204c comprising the determined overall patient health status, the generated healthcare recommendations, and the generated alerts as a part of the healthcare actionable intelligence data. An example of a code snippet of the healthcare data analysis module 1903e and the report generation module 204b executed by the processor 1901 of the HAIDGDS 204 for determining overall patient health status, generating healthcare recommendations and alerts for the identified patient 205, and generating the healthcare actionable intelligence report 204c is disclosed below:

```
public IEnumerable<MedicationAlertCatVM> GetMedicationAlertCategory(decimal?
pd_member_id_rx, decimal pd_mr_id_rx = −1)
{
  DateTime birthday =
  unitOfWork.MEMBERsRepository.GetByID(pd_member_id_rx).MBR.DOB.Val
  ue;
  if (pd_mr_id_rx == −1)
  {
    pd_mr_id_rx = unitOfWork.MRsRepository.Get( ).Where(w =>
    w.MEMBER_ID_RX = = pd_member_id_rx && w.MR_TYPE ==
    (decimal)EnumManager.MRType.PMLDefault && (w.DELETE_FLG !=
    “Y” || w.DELETE_FLG == null)).Select
  }
  IEnumerable<string> ndcCodes = GetNDC(pd_member_id_rx, pd_mr_id_rx);
  IEnumerable<string> ICdCodes = GetICD(pd_member_id_rx, pd_mr_id_rx);
  // need to come back after override
  var lstDt = (from dt in unitOfWork.DTP_DETAILRepository.Get( )
      join rc in unitOfWork.DTP_RECOMMENDATIONRepository.Get( )
      on dt.DTP_RECOMM_ID_RX equals rc.DTP_RECOMM_ID_RX
      where rc.MEMBER_ID_RX == pd_member_id_rx
        && (dt.DELETE_FLG ! = “Y” || dt.DELETE_FLG == null)
        && (rc. THERAP_ALERT_ACTION ==
        (decimal)EnumManager.TherapAlertAction.Override)
        && (rc.THERAP_ALERT_OVR_PD ! = null)
      select new { dt.NDC, dt.NDC2, dt.DISEASE_ID_RX,
      rc.THERAP_ALERT_OVR_PD, dt.INTERACTION_TEXT,
      dt.ALLERGY_ID_RX }).Union(
      from dt in unitOfWork.DTP_DETAILRepository.Get( )
      join rc in unitOfWork.DTP_RECOMMENDATIONRepository.Get( )
      on dt.DTP_RECOMM_ID_RX equals rc.DTP_RECOMM_ID_RX
      join cm in unitOfWork.DTP_COMMUNICATIONRepository.Get( )
      on rc.DTP_RECOMM_ID_RX equals cm.DTP_RECOMM_ID_RX
      join rs in unitOfWork.DTP_RESOLUTIONRepository.Get( ).Where(d
      => d.DTP_RESOLUTION_TYPE != null &&
      d.DTP_RESOLUTION_TYPE != null)
      on cm.DTP_COMM_ID_RX equals rs.DTP_COMM_ID_RX
      where rc.MEMBER_ID_RX == pd_member_id_rx
      select new {dt.NDC, dt.NDC2, dt.DISEASE_ID_RX,
      rc.THERAP_ALERT_OVR_PD, dt.INTERACTION_TEXT,
      dt.ALLERGY_ID_RX }}.Distinct ( );
  IEnumerable<MedicationAlertVM>1stMedAlertMinSevCode =
  unitOfWork.VM_DRUG_INTERACTIONRespository.Get( )
          .Where(w => ndcCodes.Contains(w.NDC1)
            && ndcCodes.Contains(w.NDC2)
            && !lstDt.Any(p => (p.NDC == w.NDC1 &&
            p.NDC2 == w.NDC2) || (p.NDC2 == w.NDC1 &&
            p.NDC == w.NDC2))
            && (w.DDI_SL == “1” || w.DDI_SL == “2”)
            )
          .Select(r => new MedicationAlertVM
          {
            ObjectIdRX = r.OBJECT_ID_RX,
            SeverityLvl = r.DDI_SL
          }). Distinct ( )
          .Union(unitOfWork.VM_DRUG_FOODRepository.Get( )
          .Where(w => ndcCodes.Contains(w.NDC) &&
          w.TXTCDE == “T”
          && !lstDt.Any(p => p.NDC == w.NDC &&
          p.INTERACTION_TEXT ==
          w.FDTXT.Replace(“MONOGRAPH TITLE:“,””))
          && (w.FD_SL == “1” || w.FD_SL == “2”)
          )
          .Select(r => newMedicationAlertVM
          {
            ObjectIdRX = r.OBJECT_ID_RX,
            SeverityLvl = r.FD_SL
          }).Distinct( )
.Union(unitOfWork.VM_MEMBER_DRUG_ALLERGYRepository.Get( )
.Where(w => w.MEMBER_ID_RX == pd_member_id_rx &&
w.ALERT_MSG.StartsWith(“Alert”) && w.MR_ID_RX == pd_mr_id_rx &&
!1stDT.Any(p => p.NDC == w.NDC && p.ALLERGY_ID_RX ==
w.ALLERGY_ID_RX)
)
.Select(r => new MedicationAlertVM
{
  ObjectiveIdRX = r.OBJECT_ID_RX,
  SeverityLvl = “1” // get this in View after letting Boris know
}
  ).Distinct( )
)
.Union(unitOfWork.VM_DRUG_DISEASERepository.Get( )
.Where(w=> ndcCodes.Contains(w.NDC)
  && ICdCodes.Contains(w.SEARCH_ICD_CD)
    && !lstDt.Any(p => p.NDC == w.NDC && p.DISEASE_ID_RX ==
    w.DISEASE_ID_RX)
    && (w.DDXCN_SL == “1” || w.DDXCN_SL == “2”)
)
.Select(r=> new MedicationAlertVM
{
  ObjectiveIdRX = r.OBJECT_ID_RX,
  SeverityLvl = r.DDXCN_SL
}).Distinct( )
)
.Union(unitOfWork.VM_DRUG_GERIATRICRepository.Get( )
.Where(w => ndcCodes.Contains(w.NDC)
  && !1stDt.Any(p => p.NDC == w.NDC)
    && ((DateTime.Now.Year − birthday.Year) * 372 + (DateTime.Now.Month −
      birthday.Month) * 31 + (DateTime.Now.Day - birthday.Day)) / 372 > 65
    &&(w.GERI_SL == “1” || w.GERI_SL == “2”)
  )
  .Select(r => new MedicationAlertVM
  {
      ObjectIdRX = r.OBJECT_ID_RX,
      SeverityLvl = r.GERI_SL
      }).Distinct( )
      )
)
.GroupBy(g=>g.ObjectIdRX)
.Select(fr=>new MedicationAlertVM
{
  ObjectIdRX= fr.Key,
  SeverityLvl = fr.Min(m => m.SeverityLvl)
});
IEnumerable<MedicationAlertCatVM> medAlertcatVM =
unitOfWork.PBM_ LOOKUP_REF_RXRepository.Get( )
        .Join(1stMedAlertMinSevCode,
          c => c.LOOKUP_REF_RX_ID,
          m => m.ObjectIdRX,
          (c, m) => new MedicationAlertCatVM
          {
            AlertCatTypeId = m.ObjectIdRX,
            AlertCatType = c.LOOKUP_VALUE,
            MinSevLvl = m.SeverityLvl
          });
return medAlertcatVM;
}
```

The masking module 1903g in communication with the report generation module 204b selectively masks the healthcare actionable intelligence data in the healthcare actionable intelligence report 204c to protect the identity of the identified patient 205. The messaging module 1903f generates a secure report access link with active session login information to access the healthcare actionable intelligence report 204c. The messaging module 1903f distributes the secure report access link to the requesting entities via the communication network 206 using one or more data exchange protocols as disclosed in the detailed description of FIG. 1. An example of a code snippet of the masking module 1903g and the messaging module 1903f executed by the processor 1901 of the healthcare actionable intelligence data generation and distribution system (HAIDGDS) 204 for selectively masking the healthcare actionable intelligence data in the healthcare actionable intelligence report 204c and for generating and distributing the secure report access link with active session login information to access the healthcare actionable intelligence report 204c to the requesting entities via the communication network 206 one or more data exchange protocols respectively is disclosed below:

```
//Get 270 X12 Parsing done
_logger.AddLogMessage(“Calling Request Parser” + Environment.NewLine);
RequestParserOutput requestParserOutput =
_iX12TOXML.DOX12TOXML(requestEDI);
if (!requestParserOutput.Is999Raised)
{
  _logger.AddLogMessage(“Request EDI Parsing Completed, Request XML is :” +
  Environmnet.NewLine);
  _logger.AddLogMessage(requestParserOutput.RequestXML +
  Environment.NewLine);
  if (requestParserOutput.RequestTranslationType = = “270”)
  {
    // Send 270 XML to DB & Collect Response from DB
    _logger.AddLogMessage(“Sending 270 Request XML For Database
    Processing: ” + Environment.NewLine);
    _responseXML =
    _prescriptionDAO.LoadEligibilityBenefitInquiry(requestParserOutput.Re
    questXML);
    _logger.AddLogMessage(“Request XML Processed in Database,
    Response XML is : ” + Environment.NewLine);
    _logger.AddLogMessage(_repsonseXML + Environment.NewLine);
  }
  else
  {
    // Send 999 XML to DB & Collect Response from DB
    _logger.AddLogMessage(“Sending 999 Request XML For Database
    Processing : ” + Environment.NewLine);
    TR999Response tR999Response =
    _prescriptionDAO.Load999ACK(requestParserOutput.RequestXML,
    requestParserOutput.GroupControlNumber);
    _logger.AddLogMessage(“Request XML Processed in Database,
    Response XML is : ” + Environment.NewLine);
    _logger.AddLogMessage(tR999Response.Ack_Header_Id +
    Environment.NewLine);
    _responseXML = “”
  }
  // Parse Response XML back to X12
  _logger.AddLogMessage(“Calling Response Parser” + Environment.NewLine);
  responseEDI = iXMLTOX12.DOXMLTOX12(_responseXML,
  requestParserOutput.RequestTransationType);
  _logger.AddLogMessage(“Response XML Parsing Completed, Response EDI is:”
  + Environment.NewLine);
  _logger.AddLogMessage(responseEDI + Environment.NewLine);
  // Return response X12
}
else
{
  _logger.AddLogMessage(“999 response being returned” +
  Environmnet.NewLine);
  responseEDI = requestParserOutput.EDI999_Response_to_270;
  _logger.AddLogMessage(“999 EDI is :” + Environment.NewLine +
  requestParserOutput.EDI999 Response_to_270);
}
}
return responseEDI;
}
```

The database 1903h of the healthcare actionable intelligence data generation and distribution system (HAIDGDS) 204 stores the retrieved and compiled healthcare data sets and the healthcare actionable intelligence data generated by the HAIDGDS 204. The modules 1903a, 1903b, 1903c, 1903d, 1903e, 204b, 1903f, 1903g, etc., of the HAIDGDS 204 communicate with the database 1903h for storing the retrieved and compiled healthcare data sets and the healthcare actionable intelligence data. In an embodiment, the database 1903h also stores the healthcare actionable intelligence report 204c. In an embodiment, the database 1903h can be any storage area or medium that can be used for storing data and files. In an embodiment, the database 1903h can be, for example, any of a structured query language (SQL) data store or a not only SQL (NoSQL) data store such as the Microsoft® SQL Server®, the Oracle® servers, the MySQL® database of MySQL AB Company, the mongoDB® of MongoDB, Inc., the Neo4j graph database of Neo Technology Corporation, the Cassandra database of the Apache Software Foundation, the HBase® database of the Apache Software Foundation, etc. In an embodiment, the database 1903h can also be a location on a file system of the HAIDGDS 204. In another embodiment, the database 1903h can be external databases remotely accessed by the HAIDGDS 204 via the communication network 206. In another embodiment, the database 1903h is configured as a cloud based database implemented in a cloud computing environment, where computing resources are delivered as a service over the communication network 206.

Computer applications and programs are used for operating the healthcare actionable intelligence data generation and distribution system (HAIDGDS) 204. The programs are loaded onto the fixed media drive 1908 and into the memory unit 1903 of the HAIDGDS 204 via the removable media drive 1909. In an embodiment, the computer applications and programs are loaded into the memory unit 1903 directly via the communication network 206. Computer applications and programs are executed by double clicking a related icon displayed on the display unit 1902 using one of the input devices 1907. The processor 1901 executes an operating system, for example, the Linux® operating system, the Unix® operating system, any version of the Microsoft® Windows® operating system, the Mac OS of Apple Inc., the IBM® OS/2, VxWorks® of Wind River Systems, Inc., QNX Neutrino® developed by QNX Software Systems Ltd., the Palm OS®, the Solaris operating system developed by Sun Microsystems, Inc., the Android® operating system of Google LLC, the Windows Phone® operating system of Microsoft Corporation, the BlackBerry® operating system of BlackBerry Limited, the iOS operating system of Apple Inc., the Symbian™ operating system of Symbian Foundation Limited, etc. The HAIDGDS 204 employs the operating system for performing multiple tasks. The operating system is responsible for management and coordination of activities and sharing of resources of the HAIDGDS 204. The operating system further manages security of the HAIDGDS 204, peripheral devices connected to the HAIDGDS 204, and network connections. The operating system employed on the HAIDGDS 204 recognizes, for example, inputs provided by a user of the HAIDGDS 204 using one of the input devices 1907, the output devices 1910, files, and directories stored locally on the fixed media drive 1908. The operating system on the HAIDGDS 204 executes different programs using the processor 1901. The processor 1901 and the operating system together define a computer platform for which application programs in high level programming languages are written.

The processor 1901 retrieves instructions defined by the data reception module 1903a, the patient identification module 1903b, the healthcare data compiler 1903c, the healthcare data storage and transformation module 1903d, the healthcare data analysis module 1903e, the report generation module 204b, the messaging module 1903f, and the masking module 1903g of the healthcare actionable intelligence data generation and distribution system (HAIDGDS) 204, for performing respective functions disclosed above. The processor 1901 retrieves instructions for executing the modules, for example, 1903a, 1903b, 1903c, 1903d, 1903e, 204b, 1903f, 1903g, etc., of the HAIDGDS 204 from the memory unit 1903. A program counter determines the location of the instructions in the memory unit 1903. The program counter stores a number that identifies the current position in the program of each of the modules, for example, 1903a, 1903b, 1903c, 1903d, 1903e, 204b, 1903f, 1903g, etc., of the HAIDGDS 204. The instructions fetched by the processor 1901 from the memory unit 1903 after being processed are decoded. The instructions are stored in an instruction register in the processor 1901. After processing and decoding, the processor 1901 executes the instructions, thereby performing one or more processes defined by those instructions.

At the time of execution, the instructions stored in the instruction register are examined to determine the operations to be performed. The processor 1901 then performs the specified operations. The operations comprise arithmetic operations and logic operations. The operating system performs multiple routines for performing a number of tasks required to assign the input devices 1907, the output devices 1910, and the memory unit 1903 for execution of the modules, for example, 1903a, 1903b, 1903c, 1903d, 1903e, 204b, 1903f, 1903g, etc., of the healthcare actionable intelligence data generation and distribution system (HAIDGDS) 204. The tasks performed by the operating system comprise, for example, assigning memory to the modules, for example, 1903a, 1903b, 1903c, 1903d, 1903e, 204b, 1903f, 1903g, etc., of the HAIDGDS 204 and to data used by the HAIDGDS 204, moving data between the memory unit 1903 and disk units, and handling input/output operations. The operating system performs the tasks on request by the operations and after performing the tasks, the operating system transfers the execution control back to the processor 1901. The processor 1901 continues the execution to obtain one or more outputs. The outputs of the execution of the modules, for example, 1903a, 1903b, 1903c, 1903d, 1903e, 204b, 1903f, 1903g, etc., of the HAIDGDS 204 are displayed to a user of the HAIDGDS 204 on the display unit 1902 via the graphical user interface 1902a and/or through the output devices 1910.

For purposes of illustration, the detailed description refers to the healthcare actionable intelligence data generation and distribution system (HAIDGDS) 204 being run locally as a single computer system; however the scope of the method and the system 200 disclosed herein is not limited to the HAIDGDS 204 being run locally as a single computer system via the operating system and the processor 1901, but may be extended to run remotely over the communication network 206 by employing a web browser and a remote server, a mobile phone, or other electronic devices. In an embodiment, one or more portions of the HAIDGDS 204 are distributed across one or more computer systems (not shown) coupled to the communication network 206.

The non-transitory computer readable storage medium disclosed herein stores computer program codes comprising instructions executable by at least one processor 1901 for securely generating and distributing healthcare actionable intelligence data to requesting entities in a computing environment. The computer program codes implement processes of various embodiments disclosed above. The computer program codes comprise a first computer program code for receiving a healthcare eligibility request from one or more of the requesting entities; a second computer program code for identifying a patient 205 from the received healthcare eligibility request; a third computer program code for retrieving and compiling healthcare data sets of the identified patient 205 from multiple healthcare data sources 401 comprising precompiled data sources via the secure data import electronic connectivity mode 402; a fourth computer program code for storing and transforming the retrieved and compiled healthcare data sets into a unified data structure 204e; a fifth computer program code for determining overall patient health status and generating healthcare recommendations and alerts for the identified patient 205 by analyzing healthcare data contained in the unified data structure 204e comprising a repository of preexisting and ongoing healthcare data sets; a sixth computer program code for generating a healthcare actionable intelligence report 204c comprising the determined overall patient health status, the generated healthcare recommendations, and the generated alerts as a part of the healthcare actionable intelligence data; and a seventh computer program code for generating and distributing a secure report access link with active session login information to access the generated healthcare actionable intelligence report 204c to one or more of the requesting entities via the communication network 206 using one or more of multiple data exchange protocols. In an embodiment, the sixth computer program code comprises an eighth computer program code for selectively masking the healthcare actionable intelligence data in the healthcare actionable intelligence report 204c to protect the identity of the identified patient 205.

The computer program codes further comprise one or more additional computer program codes for performing additional steps that may be required and contemplated for securely generating and distributing healthcare actionable intelligence data to requesting entities in a computing environment. In an embodiment, a single piece of computer program code comprising computer executable instructions performs one or more steps of the method disclosed herein for securely generating and distributing healthcare actionable intelligence data to requesting entities in a computing environment. The computer program codes comprising computer executable instructions are embodied on the non-transitory computer readable storage medium. The processor 1901 of the healthcare actionable intelligence data generation and distribution system (HAIDGDS) 204 retrieves these computer executable instructions and executes them. When the computer executable instructions are executed by the processor 1901, the computer executable instructions cause the processor 1901 to perform the steps of the method for securely generating and distributing healthcare actionable intelligence data to requesting entities in a computing environment.

It is apparent in different embodiments that the various methods, algorithms, and computer programs disclosed herein are implemented on non-transitory computer readable storage media appropriately programmed for computing devices. The non-transitory computer readable storage media participate in providing data, for example, instructions that are read by a computer, a processor or a similar device. In different embodiments, the “non-transitory computer readable storage media” also refer to a single medium or multiple media, for example, a centralized database, a distributed database, and/or associated caches and servers that store one or more sets of instructions that are read by a computer, a processor or a similar device. The “non-transitory computer readable storage media” also refer to any medium capable of storing or encoding a set of instructions for execution by a computer, a processor or a similar device and that causes a computer, a processor or a similar device to perform any one or more of the methods disclosed herein. Common forms of the non-transitory computer readable storage media comprise, for example, a floppy disk, a flexible disk, a hard disk, magnetic tape, a laser disc, a Blu-ray Disc® of the Blu-ray Disc Association, any magnetic medium, a compact disc-read only memory (CD-ROM), a digital versatile disc (DVD), any optical medium, a flash memory card, punch cards, paper tape, any other physical medium with patterns of holes, a random access memory (RAM), a programmable read only memory (PROM), an erasable programmable read only memory (EPROM), an electrically erasable programmable read only memory (EEPROM), a flash memory, any other memory chip or cartridge, or any other medium from which a computer can read.

In an embodiment, the computer programs that implement the methods and algorithms disclosed herein are stored and transmitted using a variety of media, for example, the computer readable media in various manners. In an embodiment, hard-wired circuitry or custom hardware is used in place of, or in combination with, software instructions for implementing the processes of various embodiments. Therefore, the embodiments are not limited to any specific combination of hardware and software. The computer program codes comprising computer executable instructions can be implemented in any programming language. Examples of programming languages that can be used comprise C, C++, C#, Java®, JavaScript®, Fortran, Ruby, Perl®, Python®, Visual Basic®, hypertext preprocessor (PHP), Microsoft® .NET, Objective-C®, etc. Other object-oriented, functional, scripting, and/or logical programming languages can also be used. In an embodiment, the computer program codes or software programs are stored on or in one or more mediums as object code. In another embodiment, various aspects of the method and the healthcare actionable intelligence data generation and distribution system (HAIDGDS) 204 disclosed herein are implemented in a non-programmed environment comprising documents created, for example, in a hypertext markup language (HTML), an extensible markup language (XML), or other format that render aspects of the graphical user interface (GUI) 1902a or perform other functions, when viewed in a visual area or a window of a browser program. In another embodiment, various aspects of the method and the HAIDGDS 204 disclosed herein are implemented as programmed elements, or non-programmed elements, or any suitable combination thereof.

Where databases are described such as the database 1903h, it will be understood by one of ordinary skill in the art that (i) alternative database structures to those described may be employed, and (ii) other memory structures besides databases may be employed. Any illustrations or descriptions of any sample databases disclosed herein are illustrative arrangements for stored representations of information. In an embodiment, any number of other arrangements are employed besides those suggested by tables illustrated in the drawings or elsewhere. Similarly, any illustrated entries of the databases represent exemplary information only; one of ordinary skill in the art will understand that the number and content of the entries can be different from those disclosed herein. In another embodiment, despite any depiction of the databases as tables, other formats including relational databases, object-based models, and/or distributed databases are used to store and manipulate the data types disclosed herein. Object methods or behaviors of a database can be used to implement various processes such as those disclosed herein. In another embodiment, the databases are, in a known manner, stored locally or remotely from a device that accesses data in such a database. In embodiments where there are multiple databases in the healthcare actionable intelligence data generation and distribution system (HAIDGDS) 204, the databases are integrated to communicate with each other for enabling simultaneous updates of data linked across the databases, when there are any updates to the data in one of the databases.

The method and the healthcare actionable intelligence data generation and distribution system (HAIDGDS) 204 disclosed herein can be configured to work in a network environment comprising one or more computers that are in communication with one or more devices via the communication network 206. In an embodiment, the computers communicate with the devices directly or indirectly, via a wired medium or a wireless medium such as the Internet, a local area network (LAN), a wide area network (WAN) or the Ethernet, a token ring, or via any appropriate communications mediums or combination of communications mediums. Each of the devices comprises processors, examples of which are disclosed above, that are adapted to communicate with the computers. In an embodiment, each of the computers is equipped with a network communication device, for example, a network interface card, a modem, or other network connection device suitable for connecting to the communication network 206. Each of the computers and the devices executes an operating system, examples of which are disclosed above. While the operating system may differ depending on the type of computer, the operating system provides the appropriate communications protocols to establish communication links with the communication network 206. Any number and type of machines may be in communication with the computers.

The method and the healthcare actionable intelligence data generation and distribution system (HAIDGDS) 204 disclosed herein are not limited to a particular computer system platform, processor, operating system, or network. In an embodiment, one or more aspects of the method and the HAIDGDS 204 disclosed herein are distributed among one or more computer systems, for example, servers configured to provide one or more services to one or more client computers, or to perform a complete task in a distributed system. For example, one or more aspects of the method and the HAIDGDS 204 disclosed herein are performed on a client-server system that comprises components distributed among one or more server systems that perform multiple functions according to various embodiments. These components comprise, for example, executable, intermediate, or interpreted code, which communicate over the communication network 206 using a communication protocol. The method and the HAIDGDS 204 disclosed herein are not limited to be executable on any particular system or group of systems, and are not limited to any particular distributed architecture, network, or communication protocol.

The foregoing examples have been provided merely for explanation and are in no way to be construed as limiting of the method and the system 200 disclosed herein. While the method and the system 200 have been described with reference to various embodiments, it is understood that the words, which have been used herein, are words of description and illustration, rather than words of limitation. Furthermore, although the method and the system 200 have been described herein with reference to particular means, materials, and embodiments, the method and the system 200 are not intended to be limited to the particulars disclosed herein; rather, the method and the system 200 extend to all functionally equivalent structures, methods and uses, such as are within the scope of the appended claims. While multiple embodiments are disclosed, it will be understood by those skilled in the art, having the benefit of the teachings of this specification, that method and the system 200 disclosed herein are capable of modifications and other embodiments may be effected and changes may be made thereto, without departing from the scope and spirit of the method and the system 200 disclosed herein.

We claim:

1. A method for securely generating and distributing healthcare actionable intelligence data to requesting entities in a computing environment, said method employing a healthcare actionable intelligence data generation and distribution system comprising at least one processor configured to execute computer program instructions for performing said method comprising:
receiving a healthcare eligibility request from one or more of said requesting entities by said healthcare actionable intelligence data generation and distribution system;
identifying a patient from said received healthcare eligibility request by said healthcare actionable intelligence data generation and distribution system;
retrieving and compiling healthcare data sets of said identified patient from a plurality of healthcare data sources comprising precompiled data sources by said healthcare actionable intelligence data generation and distribution system via a secure electronic connectivity mode;
storing and transforming said retrieved and compiled healthcare data sets into a unified data structure by said healthcare actionable intelligence data generation and distribution system;
determining overall patient health status and generating healthcare recommendations and alerts for said identified patient by said healthcare actionable intelligence data generation and distribution system by analyzing healthcare data contained in said unified data structure comprising a repository of preexisting and ongoing healthcare data sets;
generating a healthcare actionable intelligence report comprising said determined overall patient health status, said generated healthcare recommendations, and said generated alerts as a part of said healthcare actionable intelligence data by said healthcare actionable intelligence data generation and distribution system; and
generating and distributing a secure report access link with active session login information to access said generated healthcare actionable intelligence report to said one or more of said requesting entities by said healthcare actionable intelligence data generation and distribution system via a communication network using one or more of a plurality of data exchange protocols.

2. The method of claim 1, wherein said secure electronic connectivity mode for said retrieval of said healthcare data sets of said identified patient from said healthcare data sources is a secure electronic data interchange connectivity mode.

3. The method of claim 1, further comprising selectively masking said healthcare actionable intelligence data in said healthcare actionable intelligence report by said healthcare actionable intelligence data generation and distribution system to protect identity of said identified patient.

4. The method of claim 1, wherein said healthcare data contained in said unified data structure comprises demographic information of said identified patient, health information of said identified patient, pharmacy medication history of said identified patient, pharmacy medication plan adherence history of said identified patient, mental health records, drug utilization data, physical vitals, patient encounter data, medical diagnosis codes, medical procedure codes, financial codes, pharmacy national drug codes, and financial transaction data.

5. The method of claim 1, wherein said healthcare actionable intelligence report further comprises one or more of daily supply information required to perform a comprehensive drug utilization review analysis, fill numbers required to perform a comprehensive adherence analysis for medication therapy management, compounding elements to determine a dispensing type of a claim, a service type for performing a detailed cost analysis, submission clarification codes for determining drug-disease related information, potential drug interaction warnings, alternative drug options, potential gaps in care, health statements, notes, industry best practices for managing an active health condition of said identified patient, and a member incurred cost for performing said detailed cost analysis.

6. The method of claim 1, wherein said retrieved healthcare data sets are of a plurality of formats comprising a health insurance portability and accountability act standard format, a national council for prescription drug programs standard format, and open source, industry standard and custom electronic data interchange formats.

7. The method of claim 1, wherein said requesting entities comprise individual healthcare providers, healthcare provider organizations, seekers of pharmacy benefit, payers, prescribers, specialists, pharmacies, claim processing switches, pharmacy claim processors, coordination of benefits facilitators, financial transaction facilitators, third party administrators, centers for medicare and medicaid services, plan sponsors, plan sponsor case and care management teams, electronic data interchange vendors, and healthcare data facilitators.

8. The method of claim 1, wherein said healthcare data sources comprise data sources of said requesting entities, health data sources, vision data sources, pharmacy data sources, dental data sources, patient vitals data sources, electronic medical records, electronic health records, personal medical records, personal health records, practice management systems, electronic prescription software, pharmacy benefit management systems, laboratory data sources, mental health data, medical claims data warehouse systems, and patient encounter data sources.

9. The method of claim 1, wherein said data exchange protocols comprise a hypertext transfer protocol, a secure hypertext transfer protocol, Winsock, a recommended standard number 232 protocol, a file transfer protocol, a virtual private network protocol, and a secure file transfer protocol.

10. A healthcare actionable intelligence data generation and distribution system for securely generating and distributing healthcare actionable intelligence data to requesting entities in a computing environment, said healthcare actionable intelligence data generation and distribution system comprising:
a non-transitory computer readable storage medium configured to store computer program instructions defined by modules of said healthcare actionable intelligence data generation and distribution system; and
at least one processor communicatively coupled to said non-transitory computer readable storage medium, said at least one processor configured to execute said computer program instructions defined by said modules of said healthcare actionable intelligence data generation and distribution system, said modules comprising:
a data reception module for receiving a healthcare eligibility request from one or more of said requesting entities;
a patient identification module for identifying a patient from said received healthcare eligibility request;
a healthcare data compiler for retrieving and compiling healthcare data sets of said identified patient from a plurality of healthcare data sources comprising precompiled data sources via a secure electronic connectivity mode;
a healthcare data storage and transformation module for storing and transforming said retrieved and compiled healthcare data sets into a unified data structure;
a healthcare data analysis module for determining overall patient health status and generating healthcare recommendations and alerts for said identified patient by analyzing healthcare data contained in said unified data structure comprising a repository of preexisting and ongoing healthcare data sets;
a report generation module for generating a healthcare actionable intelligence report comprising said determined overall patient health status, said generated healthcare recommendations, and said generated alerts as a part of said healthcare actionable intelligence data; and
a messaging module for generating and distributing a secure report access link with active session login information to access said generated healthcare actionable intelligence report to said one or more of said requesting entities via a communication network using one or more of a plurality of data exchange protocols.

11. The healthcare actionable intelligence data generation and distribution system of claim 10, wherein said modules further comprise a masking module in communication with said report generation module for selectively masking said healthcare actionable intelligence data in said healthcare actionable intelligence report to protect identity of said identified patient.

12. The healthcare actionable intelligence data generation and distribution system of claim 10, wherein said healthcare data contained in said unified data structure comprises demographic information of said identified patient, health information of said identified patient, pharmacy medication history of said identified patient, pharmacy medication plan adherence history of said identified patient, mental health records, drug utilization data, physical vitals, patient encounter data, medical diagnosis codes, medical procedure codes, financial codes, pharmacy national drug codes, and financial transaction data.

13. The healthcare actionable intelligence data generation and distribution system of claim 10, wherein said healthcare actionable intelligence report further comprises one or more of daily supply information required to perform a comprehensive drug utilization review analysis, fill numbers required to perform a comprehensive adherence analysis for medication therapy management, compounding elements to determine a dispensing type of a claim, a service type for performing a detailed cost analysis, submission clarification codes for determining drug-disease related information, potential drug interaction warnings, alternative drug options, potential gaps in care, health statements, notes, industry best practices for managing an active health condition of said identified patient, and a member incurred cost for performing said detailed cost analysis.

14. The healthcare actionable intelligence data generation and distribution system of claim 10, wherein said retrieved healthcare data sets are of a plurality of formats comprising a health insurance portability and accountability act standard format, a national council for prescription drug programs standard format, and open source, industry standard and custom electronic data interchange formats.

15. The healthcare actionable intelligence data generation and distribution system of claim 10, wherein said requesting entities comprise individual healthcare providers, healthcare provider organizations, seekers of pharmacy benefit, payers, prescribers, specialists, pharmacies, claim processing switches, pharmacy claim processors, coordination of benefits facilitators, financial transaction facilitators, third party administrators, centers for medicare and medicaid services, plan sponsors, plan sponsor case and care management teams, electronic data interchange vendors, and healthcare data facilitators.

16. The healthcare actionable intelligence data generation and distribution system of claim 10, wherein said healthcare data sources comprise data sources of said requesting entities, health data sources, vision data sources, pharmacy data sources, dental data sources, patient vitals data sources, electronic medical records, electronic health records, personal medical records, personal health records, practice management systems, electronic prescription software, pharmacy benefit management systems, laboratory data sources, mental health data, medical claims data warehouse systems, and patient encounter data sources.

17. The healthcare actionable intelligence data generation and distribution system of claim 10, wherein said data exchange protocols comprise a hypertext transfer protocol, a secure hypertext transfer protocol, Winsock, a recommended standard number 232 protocol, a file transfer protocol, a virtual private network protocol, and a secure file transfer protocol.

18. A non-transitory computer readable storage medium having embodied thereon, computer program codes comprising instructions executable by at least one processor for securely generating and distributing healthcare actionable intelligence data to requesting entities in a computing environment, said computer program codes comprising:
a first computer program code for receiving a healthcare eligibility request from one or more of said requesting entities;
a second computer program code for identifying a patient from said received healthcare eligibility request;
a third computer program code for retrieving and compiling healthcare data sets of said identified patient from a plurality of healthcare data sources comprising precompiled data sources via a secure electronic connectivity mode;
a fourth computer program code for storing and transforming said retrieved and compiled healthcare data sets into a unified data structure;
a fifth computer program code for determining overall patient health status and generating healthcare recommendations and alerts for said identified patient by analyzing healthcare data contained in said unified data structure comprising a repository of preexisting and ongoing healthcare data sets;
a sixth computer program code for generating a healthcare actionable intelligence report comprising said determined overall patient health status, said generated healthcare recommendations, and said generated alerts as a part of said healthcare actionable intelligence data; and
a seventh computer program code for generating and distributing a secure report access link with active session login information to access said generated healthcare actionable intelligence report to said one or more of said requesting entities via a communication network using one or more of a plurality of data exchange protocols.

19. The non-transitory computer readable storage medium of claim 18, wherein said sixth computer program code comprises an eighth computer program code for selectively masking said healthcare actionable intelligence data in said healthcare actionable intelligence report to protect identity of said identified patient.

20. The non-transitory computer readable storage medium of claim 18, wherein said healthcare actionable intelligence report further comprises one or more of daily supply information required to perform a comprehensive drug utilization review analysis, fill numbers required to perform a comprehensive adherence analysis for medication therapy management, compounding elements to determine a dispensing type of a claim, a service type for performing a detailed cost analysis, submission clarification codes for determining drug-disease related information, potential drug interaction warnings, alternative drug options, potential gaps in care, health statements, notes, industry best practices for managing an active health condition of said identified patient, and a member incurred cost for performing said detailed cost analysis.
